(19) United States
(12) Patent Application Publication
(10) Pub. No.: US 2025/0193514 A1
(43) Pub. Date: Jun. 12, 2025

(54) Healthy Selfie (TM) -- Methods and Systems for Improving the Accuracy of Phone Images of a Pathology or Injury on a Body

(71) Applicant: Robert A. Connor, Wyoming, MN (US)

(72) Inventor: Robert A. Connor, Wyoming, MN (US)

(73) Assignee: Holovisions LLC, Ham Lake, MN (US)

(21) Appl. No.: 19/060,730

(22) Filed: Feb. 23, 2025

Related U.S. Application Data
(63) Continuation-in-part of application No. 18/431,946, filed on Feb. 3, 2024.
Application No. 18/431,946 is a continuation-in-part of application No. 17/722,979, filed on Apr. 18, 2022, now Pat. No. 12,014,500.
Application No. 17/722,979 is a continuation-in-part of application No. 17/404,174, filed on Aug. 17, 2021, now Pat. No. 11,308,618.
Application No. 17/404,174 is a continuation-in-part of application No. 16/706,111, filed on Dec. 6, 2019, now Pat. No. 11,176,669.
(60) Provisional application No. 62/833,761, filed on Apr. 14, 2019.

(51) Int. Cl. H04N 23/60; A61B 5/00; G16H 30/40

(52) U.S. Cl. CPC H04N 23/64; A61B 5/0077; A61B 5/6898; A61B 5/742; A61B 5/7455; G16H 30/40; A61B 5/445

(57) ABSTRACT

This invention is a method, device, or system to guide a person concerning how to operate a conventional phone to record more-accurate images of a pathology or injury (e.g. such as a wound or skin lesion) on a body for remote medical evaluation purposes. In an example, machine learning and/or artificial intelligence can be used to guide the person concerning how to move a phone along a virtual arcuate path or shape in space over, across, or around the pathology or injury. In an example, this guidance can be auditory, visual, and/or tactile.

### CROSS-REFERENCE TO RELATED APPLICATIONS

This application is a continuation-in-part of U.S. patent application Ser. No. 18/431,946 which was filed on 2024 Feb. 3. U.S. patent application Ser. No. 18/431,946 was a continuation-in-part of U.S. patent application Ser. No. 17/722,979 which was filed on 2022 Apr. 18. U.S. patent application Ser. No. 17/722,979 was a continuation-in-part of U.S. patent application Ser. No. 17/404,174 which was filed on 2021 Aug. 17 and issued as U.S. patent Ser. No. 11/308,618 on 2022 Apr. 19. U.S. patent application Ser. No. 17/404,174 was a continuation-in-part of U.S. patent application Ser. No. 16/706,111 which was filed on 2019 Dec. 6 and issued as U.S. patent Ser. No. 11/176,669 on 2021 Nov. 16. U.S. patent application Ser. No. 16/706,111 claimed the priority benefit of U.S. provisional patent application 62/833,761 filed on 2019 Apr. 14. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to remote medical imaging using conventional mobile devices.

### Introduction

There are many potential benefits from remote medical evaluation (e.g. “telemedicine” or “virtual care”) via electronic communication. With remote medical evaluation, people in low-population areas who do not have local healthcare specialists can have remote access to care from non-local specialists. Also, remote medical evaluation can help people with limited transportation to receive care from their homes and help people with busy work schedules to get care at convenient times. Also, as highlighted by a recent pandemic, remote medical evaluation can help people to avoid in-person contact in medical offices which can spread contagious disease. Further, from a provider perspective, remote medical evaluation can enhance provider productivity, lower the cost of care, and broaden a provider's geographic service area.

For these reasons, as well as technological advances in phone cameras and artificial intelligence, there has been progress and growth in remote medical evaluation during the past several years. For example, the quality and functionality of internet-based medical video conferencing has improved. Mobile phones have become ubiquitous and the general quality of images captured by their cameras has improved. Increasingly-sophisticated artificial intelligence systems are playing a greater role in telemedicine and virtual care portals.

Despite these advances, however, there are still significant challenges for remote medical evaluation, especially for remote imaging such as remote assessment of wounds and skin lesions (e.g. teledermatology). Potential problems with images recorded by patients at home with conventional phones include: out-of-focus images; insufficient range of image angles to gauge three-dimensional attributes of a pathology or injury; color calibration difficulties due to different color filters and spectra in different types of phones; size calibration difficulties due to different imaging distances and angles; and improper ambient lighting levels or types. There remains an unmet need for methods and systems to guide patients at home concerning how to record more-accurate phone images of pathologies or injuries (e.g. wounds and skin lesions) for remote medical evaluation purposes. This is the need which is addressed by the invention (the “Healthy Selfie” ™) which is disclosed herein.

### REVIEW OF THE RELEVANT ART

U.S. patent application publication 20140073880 (Boucher et al., Mar. 13, 2014, “Devices, Methods and Systems for Acquiring Medical Diagnostic Information and Provision of Telehealth Services”) discloses consumer and user-friendly telemedicine systems and procedures which enable health services and/or diagnosis to be provided remotely. U.S. Pat. No. 8,755,053 (Fright et al., Jun. 17, 2014, “Method of Monitoring a Surface Feature and Apparatus Therefor”) discloses systems for determining dimensions of a surface feature by capturing an image of the surface feature and determining a scale associated with the image.

U.S. patent application publication 20140300722 (Garcia, Oct. 9, 2014, “Image-Based Measurement Tools”) discloses methods, systems, devices, and computer programs enabling the measurement of various objects using imaging. U.S. patent application publication 20140313303 (Davis et al., Oct. 23, 2014, “Longitudinal Dermoscopic Study Employing Smartphone-Based Image Registration”) discloses capturing and analyzing skin images at different times using a smartphone. U.S. patent application publication 20150119652 (Hyde et al., Apr. 30, 2015, “Telemedicine Visual Monitoring Device with Structured Illumination”) discloses a system for providing telemedicine support with structured illumination.

U.S. Pat. No. 9,377,295 (Fright et al., Jun. 28, 2016, “Method of Monitoring a Surface Feature and Apparatus Therefor”) and U.S. patent application publication 20170042452 (Fright et al., Feb. 16, 2017, “Method of Monitoring a Surface Feature and Apparatus Therefor”) disclose systems for determining dimensions of a surface feature by capturing an image of the surface feature and determining a scale associated with the image. U.S. Pat. No. 9,861,285 (Fright et al., Jan. 9, 2018, “Handheld Skin Measuring or Monitoring Device”) discloses a camera and a structured light arrangement which projects three or more laser fan beams such that the laser fan beams cross in front of the camera.

U.S. patent application publication 20180279943 (Budman et al., Oct. 4, 2018, “System and Method for the Analysis and Transmission of Data, Images and Video Relating to Mammalian Skin Damage Conditions”) discloses the collection of data, images and video characterizing mammalian skin damage conditions using a mobile device as a data collection engine at the point of care. U.S. patent application publication 20180289334 (De Brouwer et al., Oct. 11, 2018, “Image-Based System and Method for Predicting Physiological Parameters”) discloses using a neural network model, such as regression deep learning convolutional neural network, to predict a physiological parameter.

U.S. patent application publication 20180293350 (Dimov et al., Oct. 11, 2018, “Image-Based Disease Diagnostics Using a Mobile Device”) discloses a diagnostic system which performs disease diagnostic tests using an optical property modifying device and a mobile device. U.S. patent application publication 20180352150 (Purwar et al., Dec. 6, 2018, “System and Method for Guiding a User to Take a Selfie”) discloses systems and methods for improving the image quality of a selfie.

U.S. patent Ser. No. 10/362,984 (Adiri et al., Jul. 30, 2019, “Measuring and Monitoring Skin Feature Colors, Form and Size”) and U.S. patent application publication 20190290187 (Ariri et al., Sep. 26, 2019, “Measuring and Monitoring Skin Feature Colors, Form and Size”) disclose kits, diagnostic systems and methods which measure the distribution of colors of skin features by comparison to calibrated colors which are co-imaged with the skin features.

U.S. patent application publication 20200059596 (Yoo et al., Feb. 20, 2020, “Electronic Device and Control Method Thereof”) discloses an electronic apparatus with a camera and a graphic user interface (GUI) for adjusting the image capture position of the camera. U.S. patent application publication 20200126226 (Adiri et al., Apr. 23, 2020, “Utilizing Personal Communications Devices for Medical Testing”) discloses a method comprising receiving an image of a reagent pad in proximity to a colorized surface having at least one pair of colored reference elements.

U.S. patent application publication 20200211193 (Adiri et al., Jul. 2, 2020, “Tracking Wound Healing Progress Using Remote Image Analysis”) discloses image-based systems and methods for tracking healing progress of multiple adjacent wounds. U.S. patent application publication 20210142888 (Adiri et al., May 13, 2021, “Image Processing Systems and Methods for Caring for Skin Features”) discloses systems and methods for image processing of a skin feature using a mobile communications device.

U.S. patent Ser. No. 11/030,778 (Burg et al., Jun. 8, 2021, “Methods and Apparatus for Enhancing Color Vision and Quantifying Color Interpretation”) discloses selecting a first color sample in a first image, selecting a second color sample in a second image, and comparing the first color sample against the second color sample.

U.S. patent application publication 20210219907 (Fright et al., Jul. 22, 2021, “Method of Monitoring A Surface Feature and Apparatus Therefor”) discloses systems for determining dimensions of a surface feature by capturing an image of the surface feature and determining a scale associated with the image. U.S. patent application publication 20210251563 (Adiri et al., Aug. 19, 2021, “Measuring and Monitoring Skin Feature Colors, Form and Size”) discloses kits, diagnostic systems, and methods which measure the distribution of colors of skin features by comparison to calibrated colors which are co-imaged with the skin features. U.S. patent Ser. No. 11/112,406 (Pulitzer et al., Sep. 7, 2021, “System and Method for Digital Remote Primary, Secondary, and Tertiary Color Calibration Via Smart Device in Analysis of Medical Test Results”) discloses a method for providing immunoassay test results which includes determining if color values of an image of a test line are within a predetermined range.

U.S. patent Ser. No. 11/116,407 (Dickie et al., Sep. 14, 2021, “Anatomical Surface Assessment Methods, Devices and Systems”) and U.S. patent application publication 20210386295 (Dickie et al., Dec. 16, 2021, “Anatomical Surface Assessment Methods, Devices and Systems”) disclose a method of assessing a feature on a patient's skin surface by capturing an image of the patient's skin surface with a camera of a portable device. U.S. patent Ser. No. 11/250,945 (Fairbairn et al., Feb. 15, 2022, “Automatically Assessing an Anatomical Surface Feature and Securely Managing Information Related to the Same”) discloses obtaining user input and/or data generated by an image capture device to assess a surface feature or update an existing assessment of the surface feature.

U.S. patent application publication 20220215538 (Robinson et al., Jul. 7, 2022, “Automated or Partially Automated Anatomical Surface Assessment Methods, Devices and Systems”) discloses a method of assessing a feature on a patient's skin surface which includes determining outline data of the feature. U.S. patent application publication 20220215545 (Adiri et al., Jul. 7, 2022, “Cross Section Views of Wounds”) discloses using a processor to generate cross sectional views of a wound.

U.S. patent application publication 20220224876 (Matts et al., Jul. 14, 2022, “Dermatological Imaging Systems and Methods for Generating Three-Dimensional (3D) Image Models”) discloses systems and methods for generating three-dimensional (3D) image models of skin surfaces. U.S. patent application publication 20220270746 (Fairbairn et al., Aug. 25, 2022, “Automatically Assessing an Anatomical Surface Feature and Securely Managing Information Related to the Same”) discloses obtaining user input and/or data generated by an image capture device to assess a surface feature or update an existing assessment of the surface feature.

U.S. patent application publication 20220051409 (Maclellan et al., Dec. 17, 2022, “Systems and Methods for Using Artificial Intelligence for Skin Condition Diagnosis and Treatment Options”) discloses methods, systems, and storage media for determining a numerical classification of human skin color and determining a personalized treatment plan. U.S. patent Ser. No. 11/676,705 (Adiri et al., Jun. 13, 2023, “Tracking Wound Healing Progress Using Remote Image Analysis”) discloses systems and methods for tracking healing progress of multiple adjacent wounds.

U.S. patent application publication 20230181042 (Fan et al., Jun. 15, 2023, “Machine Learning Systems and Methods for Assessment, Healing Prediction, and Treatment of Wounds”) and U.S. patent application publication 20230222654 (Fan et al., Jul. 13, 2023, “Machine Learning Systems and Methods for Assessment, Healing Prediction, and Treatment of Wounds”) disclose machine learning systems and methods for prediction of wound healing, such as for diabetic foot ulcers or other wounds.

U.S. patent application publication 20230270373 (Adiri et al., Aug. 31, 2023, “Measuring and Monitoring Skin Feature Colors, Form and Size”) discloses kits, diagnostic systems, and methods which measure the distribution of colors of skin features by comparison to calibrated colors which are co-imaged with the skin features. U.S. patent Ser. No. 11/749,399 (Adiri et al., Sep. 5, 2023, “Cross Section Views of Wounds”) discloses using a processor to generate cross sectional views of a wound. U.S. patent Ser. No. 11/783,480 (Moore, Oct. 10, 2023, “Semi-Automated System for Real-Time Wound Image Segmentation and Photogrammetry on a Mobile Platform”) discloses a wound imaging system with a user interface, a computer processor, and an active contouring module.

U.S. patent Ser. No. 11/835,515 (Berg et al., Dec. 5, 2023, “Method for Evaluating Suitability of Lighting Conditions for Detecting an Analyte in a Sample Using a Camera of a Mobile Device”) discloses a method of evaluating suitability of lighting conditions for detecting an analyte in a sample using a mobile device camera. U.S. patent application publication 20240056673 (Shi, Feb. 15, 2024, “Camera Control Method and Apparatus, and Storage Medium”) discloses a camera control method which includes receiving image data from a second terminal device and sending an operation command for the photographing process of the second terminal device.

U.S. patent Ser. No. 11/903,723 (Barclay et al., Feb. 20, 2024, “Anatomical Surface Assessment Methods, Devices and Systems”) discloses methods for assessing a skin surface of a patient including receiving a three-dimensional data set representative of the patient's skin surface. U.S. patent application publication 20240087115 (Hong et al., Mar. 14, 2024, “Machine Learning Enabled System for Skin Abnormality Interventions”) discloses using a convolutional neural network to classify and measure skin abnormalities. U.S. patent application publication 20240096468 (Clark, Mar. 21, 2024, “Electronic System for Wound Image Analysis and Communication”) discloses embodiments for analysis of images of a wound and prescription information received from a mobile device.

U.S. patent Ser. No. 12/008,752 (Price, Jun. 11, 2024, “Automated Scan of Common Ailments So That a Consistent Image Can Be Given to a Doctor for Analysis”) discloses techniques for automated alignment of image capture of physical ailments. U.S. patent application publication 20240197241 (Barclay et al., Jun. 20, 2024, “Anatomical Surface Assessment Methods, Devices and Systems”) discloses methods for assessing a skin surface including receiving a three-dimensional data set. U.S. patent application publication 20240331136 (Valles Leon, Oct. 3, 2024, “Machine Learning to Predict Medical Image Validity and to Predict a Medical Diagnosis”) discloses a method which transmits an image and predictions to a healthcare provider.

U.S. patent Ser. No. 12/130,237 (Klein et al., Oct. 29, 2024, “Method for Calibrating a Camera of a Mobile Device for Detecting an Analyte in a Sample”) discloses a method for calibrating a camera of a mobile device for detecting an analyte in a sample. U.S. patent application publication 20240365002 (Chen et al., Oct. 31, 2024, “Image Capture Method, and Related Apparatus and System”) discloses an image capture method with a master device and a slave device. U.S. patent application publication 20240412373 (Maclellan et al., Dec. 12, 2024, “Systems and Methods for Using Artificial Intelligence for Skin Condition Diagnosis and Treatment Options”) discloses methods, systems, and storage media for determining a numerical classification of human skin color and determining a personalized treatment plan.

U.S. patent application publication 20240412849 (Fairbairn et al., Dec. 12, 2024, “Automatically Assessing an Anatomical Surface Feature and Securely Managing Information Related to the Same”) discloses a facility for procuring and analyzing information about an anatomical surface feature from a caregiver that is usable to generate an assessment of the surface feature. U.S. patent application publication 20250005761 (Thatcher et al., Jan. 2, 2025, “System and Method for Topological Characterization of Tissue”) discloses an imaging system with a plurality of imaging sensors configured to receive light reflected by a tissue region and generate a 3D model of the tissue region.

### SUMMARY OF THE INVENTION

This invention is a method, device, or system to guide a person concerning how to move or otherwise operate a conventional mobile phone to record more-accurate images of a pathology or injury (e.g. such as a wound or skin lesion) on a body for remote medical evaluation purposes. In an example, machine learning and/or artificial intelligence can be used to guide the person concerning how to move a phone along a virtual arcuate path or shape in space over, across, or around the pathology or injury. In an example, this guidance can be auditory, visual, and/or tactile.

In an example, a second set of images of a pathology or injury which are guided by this method, device, or system can be more accurate and/or more complete for remote medical evaluation than an unguided first set of images for one or more of the following reasons: the second set shows the pathology in sharper focus; the second set shows the pathology with greater magnification; the second set shows the pathology from a wider range of angles; the second set shows the pathology from a greater range of distances; the second set includes a larger portion of the pathology; the second set has better illumination; and the second set includes an environmental object near the pathology for calibration of color and/or size.

### BRIEF INTRODUCTION TO THE FIGURES

FIG. 1 shows an example of a method or system for recording images of a pathology (or injury) on a body by moving a phone along a virtual arcuate path or shape.

FIG. 2 shows an example of a method or system which uses auditory signals to guide a person concerning how to move a phone along a virtual arcuate path or shape to record images of a pathology (or injury) on a body.

FIG. 3 shows an example of a method or system for recording images of a pathology (or injury) on a body by moving a phone along a virtual spiral or helical path or shape.

FIG. 4 shows an example of a method or system for recording images of a pathology (or injury) on a body by moving a phone along a virtual starburst-shaped path or shape.

### DETAILED DESCRIPTION OF THE FIGURES

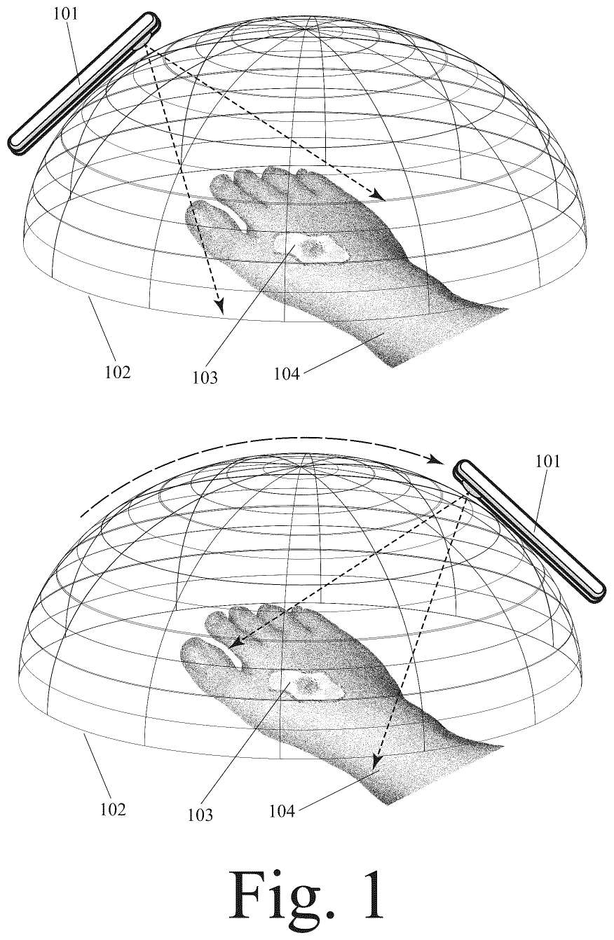

Before discussing the specific embodiments of this invention which are shown in FIGS. 1 through 4, this disclosure provides an introductory section which covers some of the general concepts, components, and methods which comprise this invention. Where relevant, these concepts, components, and methods can be applied as variations to the examples shown in FIGS. 1 through 4 which are discussed afterwards.

In an example, a method to guide a person concerning how to move or otherwise operate a phone (or other imaging device) to record images of a pathology (or injury) on a body can comprise: receiving a first set of one or more images of a pathology on a body recorded by the phone (or other imaging device); analyzing the first set of one or more images; and guiding the person concerning how to use the phone to record a second set of one or more images of the pathology.

In an example, a guided second set of images of a pathology (or injury) can be more accurate and/or more complete for medical evaluation than an unguided first set of images, for one or more of the following reasons: the second set shows the pathology in sharper focus; the second set shows the pathology with greater magnification; the second set shows the pathology from a wider range of angles; the second set shows the pathology from a greater range of distances; the second set includes a larger portion of the pathology; the second set has better illumination; and the second set includes an environmental object near the pathology for calibration of color and/or size.

In an example, the person can be guided concerning how to move or otherwise operate the phone (or other imaging device) by visual guidance comprising written words, arrows or other directional indictors, graphic symbols, animations, light emission, laser beams, and/or displayed images. In an example, the person can be guided concerning how to move or otherwise operate the phone (or other imaging device) by an augmented reality display. In an example, an augmented reality display can show the pathology (or injury) on a body, the location of the phone (or other imaging device), and how the phone should be moved to record images of the pathology.

In an example, the person can be guided concerning how to move or otherwise operate the phone (or other imaging device) by auditory guidance comprising spoken words, tones or sounds, and/or tone or sound sequences. In an example, the person can be guided concerning how to move or otherwise operate the phone (or other imaging device) by tactile and/or haptic guidance comprising vibrations and/or vibration sequences. In an example, the person can be given one or more auditory, visual, and/or tactile cues, signals, and/or communications with first characteristics if the phone (or other imaging device) should be moved closer to the pathology and the person is given one or more auditory, visual, and/or tactile cues, signals, and/or communications with second characteristics if the phone should be moved father from the pathology.

In an example, machine learning and/or artificial intelligence can be used for: analyzing the first set of one or more images; and guiding the person concerning how to move or otherwise operate the phone (or other imaging device) to record the second set of one or more images.

In an example, analysis of the first set of one or more images can include identifying a virtual path or shape in three-dimensional space which is across, over, or around the pathology along which the phone (or other imaging device) should be moved to record the second set of one or more images; and the person can be guided concerning how to move the phone along the virtual path or shape. In an example, the virtual path or shape can be a section of a circle, sphere, ellipse, or ellipsoid. In an example, the person can be guided to move the phone in an oscillating, back-and-forth, sinusoidal, serpentine, spiral, helical, or starburst pattern along the virtual path or shape.

In an example, the person can be given one or more auditory, visual, and/or tactile cues, signals, and/or communications with first characteristics if the phone (or other imaging device) is on the virtual path or shape and given one or more auditory, visual, and/or tactile cues, signals, and/or communications with second characteristics if the phone is not on the virtual path or shape.

In an example, a system to guide the person concerning how to use a phone (or other imaging device) to record images of a pathology (or injury) on a body can comprise: a phone which records images of a pathology (or injury) on a body; and an augmented reality display. In an example, machine learning and/or artificial intelligence can be used to analyze the images of the pathology (or injury). In an example, machine learning and/or artificial intelligence can be used to provide the person with guidance concerning how to move the phone (or other imaging device) to record images of the pathology (or injury).

In an example, guidance concerning how to move the phone (or other imaging device) can be provided via the augmented reality display. In an example, the augmented reality display can show the pathology, the location of the phone (or other imaging device), and how the phone should be moved. In an example, the augmented reality display can be on the screen of a second phone or other mobile device. In an example, the augmented reality display can be part of eyewear.

In an example, a pathology (or injury) can be a diabetic ulcer. In an example, a pathology (or injury) can be a skin wound. In an example, a pathology can be a skin lesion which may be skin cancer. In another example, a pathology can be skin cancer. In an example, an injury can be a skin abrasion. In an example, analysis of phone (or other imaging device) images of a pathology (or injury) on a body by machine learning and/or artificial intelligence can include evaluation of the size, area, depth, volume, shape, outline, texture, color, temperature, spectral absorption distribution, and/or movement of the pathology.

In an example, a method can comprise using machine learning and/or artificial intelligence to analyze phone (or other imaging device) images of a pathology (or injury) on a body to assist in the diagnosis, management, and/or treatment of a health condition. In another example, a method for medical evaluation of phone (or other imaging device) images of a pathology (or injury) on a body can comprise: initial analysis using machine learning and/or artificial intelligence; and follow-up analysis by human evaluation if the initial analysis indicates probable pathology.

In an example, a method can including analysis of phone (or other imaging device) images of a pathology (or injury) on a body using one or more analytical methods selected from the group consisting of: angular calibration, artificial intelligence, boundary determination, cluster analysis, color and texture analysis, color calibration, discriminant analysis, Fourier transformation, image attribute adjustment or normalization, image pattern recognition, image segmentation, linear discriminant analysis, logistic regression, machine learning, multivariate linear regression, neural network, non-linear programming, carlavian curve analysis, pattern recognition, principal components analysis, size calibration, spectral analysis, three-dimensional modeling, time series analysis, volumetric analysis, and volumetric modeling.

In an example, a method can comprise: (a) receiving a first set of one or more images of a pathology (or injury) on a body which have been recorded by a phone (or other imaging device); (b) analyzing the first set of images using machine learning and/or artificial intelligence to identify an arcuate path (or shape) in 3D space along which the phone should be moved to record a superior second set of one or more images of the pathology; and (c) generating auditory directional indicators (e.g. tone sequences or spoken directional instructions) which guide a person concerning move the phone along the identified path. In an example, a method can comprise: using machine learning and/or artificial intelligence to evaluate the usefulness of a first image of a pathology (or injury) on a body for medical evaluation purposes; and instructing a person to record a second image if the first image is not satisfactory for medical evaluation purposes.

In another example, a method for guiding a person concerning how to move a phone (or other imaging device) to record images of a pathology (or injury) on a body can comprise: receiving a first set of images of the pathology recorded by the phone; analyzing the first set of images to identify a path (or shape) in 3D space along which the phone should be moved to record a second set of images of the pathology; and guiding the person (e.g. with visual, auditory, or tactile guidance) concerning how to move the phone along this path, wherein the phone is pivoted, tilted, and/or rotated as it is moved along the path so that the focal vector of the phone camera remains pointed toward the pathology.

In an example, a method for recording images of a pathology on a body can comprise: (a) receiving a first set of one or more images of a pathology on a body which have been recorded by a phone (or other imaging device); (b) using machine learning and/or artificial intelligence to analyze the first set of one or more images and to specify a virtual arcuate path (or shape) in space from which a second set of one or more images of the pathology should be recorded; and (c) providing the person with auditory guidance concerning how to keep along the virtual arcuate path as the phone is moved to record the second set of one or more images.

In an example, a method to guide a person concerning how to move a phone (or other imaging device) to record images of a pathology (or injury) on a body can comprise: asking a person to use a phone to record a first set of one or more images of the pathology; receiving the first set of one or more images from the phone; analyzing the first set of one or more images using machine learning and/or artificial intelligence; determining a virtual path or shape in 3D space along which the phone should be moved to record a second set of one or more images which is better focused, more accurate, more comprehensive, and/or more useful for medical evaluation than the first set; and providing auditory, visual, or tactile guidance to the person concerning how to move the phone along this virtual path or shape.

In an example, a method to guide a person concerning how to move a phone (or other imaging device) to record images of a pathology (or injury) on a body can comprise: asking a person to use a phone to record a first set of one or more images of the pathology; receiving the first set of one or more images from the phone; analyzing the first set of one or more images using machine learning and/or artificial intelligence; based on analysis of the first set of one or more images, providing guidance to the person concerning how to move the phone to record a second set of one or more images of the pathology, wherein the second set is better focused, more accurate, more comprehensive, and/or more useful for medical evaluation than the first set.

In another example, a method to guide a person concerning how to move a phone (or other imaging device) to record images of a pathology (or injury) on a body can comprise: receiving a first set of one or more images of a pathology (or injury) on a body recorded by a phone (or other imaging device); analyzing the first set of one or more images using machine learning and/or artificial intelligence; and providing guidance to the person concerning how to use the phone (e.g. moving the phone or changing camera parameters) to record a second set of one or more images of the pathology, wherein the second set of one or more images enables more accurate diagnosis, characterization, and/or measurement of the pathology (or injury) than the first set of one or more images, and wherein the guidance is at least partly based on analysis of the first set of one or more images.

In an example, a system for helping a person to record high-quality images of a pathology (or injury) on a body for medical evaluation using a phone (or other imaging device) can comprise: a phone; a camera on the phone; a data processor; and a sound-generating component (e.g. speaker); wherein the data processor receives a first set of images of the pathology which has been recorded by the camera; wherein the data processor (e.g. using machine learning and/or artificial intelligence) analyzes the first set of images to determine an arcuate path (or shape) in 3D space along which the phone should be moved to record a superior second set of images of the pathology; and wherein the system uses the sound-generating component to provide a person with real-time auditory guidance (e.g. tones or spoken words) concerning how to move the phone along the arcuate path in 3D space.

In another example, analysis of a first set of images of a pathology (or injury) can include identifying a virtual path or shape in three-dimensional space which is across, over, or around the pathology along which a phone (or other imaging device) should be moved to record a superior second set of images the pathology; and a person can then be guided concerning how to move the phone along the virtual path or shape to record the superior second set of images. In an example, machine learning and/or artificial intelligence can evaluate the quality (e.g. focus, magnification, completeness, distance, angle, perspective, lighting, and/or color) of a first set of phone images of a pathology (or injury) on a body and recommend actions to a person to improve image quality for recording a second set of phone images.

In an example, a method for imaging a pathology (or injury) on a body can comprise: receiving a first set of one or more images of a pathology (or injury) on a body recorded by a phone (or other imaging device); analyzing (e.g. using machine learning and/or artificial intelligence) the first set of one or more images to determine a path (or shape) in 3D space along which a phone should be moved to record a second set of one or more images of the pathology, wherein the second set is more useful for medical evaluation (e.g. better focus, better depth estimation, better color calibration, better range for angles for 3D model creation) than the first set; and guiding a person concerning how to move the phone along the path (or shape) in 3D space to record the second set of one or more images.

In an example, a method, device, or system can use machine learning and/or artificial intelligence to guide a person concerning how to record a second (guided) set of images of a pathology (or injury) on a body which are superior to those recorded in a first (unguided) set of images because the second set shows the pathology from a wider range of angles. In an example, a second set of one or more images of a pathology (e.g. wound, ulcer, or skin lesion) on a body can correct problems with a first set of one or more images of the pathology such as poor focus, limited field of view, limited range of angles, and/or poor illumination.

In an example, a method can include guiding and/or instructing a person concerning how to move a phone (or other imaging device) to one or more different locations to record images of a pathology (or injury) on a body from different distances and/or different angles. In another example, a person with a pathology (or injury) on their body can move a phone (or other imaging device) over, across, and/or around the pathology to record images for medical evaluation. In an example, a method can comprise providing a person with guidance concerning how to move a phone (or other imaging device) so as to maintain focal direction toward the (center of) a pathology (or injury) on a body and also maintain a constant distance between the phone and the pathology.

In another example, a method can comprise providing a real-time dialog between a person moving a phone (or other imaging device) and an AI-controlled system, wherein this real-time dialog guides the person concerning how to move the phone in real-time relative to a pathology (or injury) on a body in order to capture in-focus images of the pathology from different locations and/or angles. In an example, a method for guiding a person concerning how to use a phone (or other imaging device) to record images of a pathology (or injury) on a body can comprise instructing the person to change the zoom setting of the phone. In an example, a method for using a phone (or other imaging device) to record images of a pathology (or injury) on a body can include allowing remote control of the focusing function of the phone, wherein this remote control automatically adjusts the focal distance of the phone to ensure that the pathology remains in focus as the phone is moved over, across, and/or around the pathology.

In an example, a method or system can comprise giving turning off an automatic focus function of a camera on a phone and giving control of the focus function to a remote server, device, or system. In an example, a method or system for imaging can give remote control of a camera focus function (e.g. setting) to a machine learning and/or artificial intelligence system (e.g. program).

In an example, a method or system can comprise giving turning off an automatic zoom function of a camera on a phone and giving control of the zoom function to a remote server, device, or system. In an example, a method or system for imaging can give remote control of a camera zoom function (e.g. setting) to a machine learning and/or artificial intelligence system (e.g. program).

In an example, a method or system can comprise giving turning off an automatic magnification function of a camera on a phone and giving control of the magnification function to a remote server, device, or system. In an example, a method or system for imaging can give remote control of a camera magnification function (e.g. setting) to a machine learning and/or artificial intelligence system (e.g. program).

In an example, a method can comprise guiding a person concerning how to move a phone (or other imaging device) in directions relative to a pathology (or injury) on a body which is being imaged (e.g. closer to the pathology, farther from the pathology, to the right of the pathology, to the left of the pathology). In an example, a method can include providing guidance and/or instruction to a person concerning how to move a phone (or other imaging device) to one or more different locations to record images of a pathology (or injury) on a body from different distances and/or different angles, wherein this guidance and/or instruction includes the distances that phone should be moved.

In an example, a method for guiding a person concerning how to move a phone (or other imaging device) over, across, and/or around a pathology (or injury) on a body can comprise: guiding the person moving the phone to perform multiple series of imaging sequences to record images of the pathology from different angles and distances; wherein each imaging sequence includes (a) positioning the plane of the phone orthogonally to a radial vector which extends out from the pathology and (b) moving the phone closer to and/or farther from the pathology along this radial vector (e.g. keeping the plane of the phone orthogonal to the radial vector).

In another example, a method to guide a person concerning how to move a phone (or other imaging device) to record images of a pathology (or injury) on a body can comprise emission of sound with a first frequency or intensity when the phone is too far from the pathology, emission of sound with a second frequency or intensity when the phone is the proper distance from the pathology, and emission of sound with a third frequency or intensity when the phone is too close to the pathology. In another example, a method, device, or system can provide a person with real-time AI-generated visual guidance concerning how to move a phone (or other imaging device) along a specified path (or shape) in 3D space over, across, and/or around a pathology (or injury) on a body, wherein a first visual pattern guides the person to move the phone closer to the pathology and a second visual pattern guides the person to move the phone farther from the pathology.

In an example, guidance concerning how to move a phone (or other imaging device) to record a set of images of a pathology (or injury) can include instructions to change the distance between the pathology (or injury) and the phone. In an example, sound emitted by a phone (or other imaging device) can have a faster pulse rate when the phone is too far from a pathology for accurate (e.g. in focus) imaging and can have a slower pulse rate when the phone is the correct distance from the pathology for accurate (e.g. in focus) imaging. In an example, sound emitted by a phone (or other imaging device) can have a higher pitch when the phone is too close to a pathology for accurate (e.g. in focus) imaging and can have a lower pitch when the phone is the correct distance from the pathology for accurate (e.g. in focus) imaging.

In an example, sound emitted by a phone (or other imaging device) can have a slower pulse rate when the phone is too far from a pathology for accurate (e.g. in focus) imaging and can have a faster pulse rate when the phone is the correct distance from the pathology for accurate (e.g. in focus) imaging. In another example, sound emitted by a phone (or other imaging device) can be louder when the phone is too close to a pathology for accurate (e.g. in focus) imaging and can be quieter (or even silent) when the phone is the correct distance from the pathology for accurate (e.g. in focus) imaging.

In an example, a method can comprise guiding a person to rotate or pivot a phone (or other imaging device) in directions relative to a pathology (or injury) on a body which is being imaged (e.g. rotate clockwise, rotate counter clockwise, rotate forward, rotate backward). In another example, a method can include providing a person with guidance concerning how to move a phone (or other imaging device) to record images of a pathology (or injury) on a body for medical evaluation, wherein this guidance comprises sound or tone sequences, and wherein different sound or tone sequences indicate different directions or orientations. In an example, a method for guiding a person concerning how to move a phone (or other imaging device) to record images of a pathology (or injury) on a body can comprise displaying different graphic symbols or symbol sequences which indicate that the phone should be moved in different directions or rotated to different orientations.

In an example, a method for guiding a person concerning how to move a phone (or other imaging device) to record images of a pathology (or injury) on a body can comprise providing auditory guidance (e.g. spoken words, tones, or tone sequences) instructing the person in which direction, or to which orientation, the person should move the phone. In an example, a method for guiding a person concerning how to move a phone (or other imaging device) to record images of a pathology (or injury) on a body can comprise instructing the person to pivot or rotate the phone (e.g. to change the phone's orientation relative to the pathology). In an example, a method for recording phone (or other imaging device) images of a pathology (or injury) on a body for medical evaluation can comprise guiding a person concerning how to pan, rotate, pivot, and/or tilt the phone to keep the phone's camera focal vector directed toward the same location on a pathology (or injury) while the phone is moved over, across, and/or around the pathology.

In an example, a method, device, or system can provide a person with real-time AI-generated auditory guidance concerning how to move a phone (or other imaging device) along a specified path (or shape) in 3D space over, across, and/or around a pathology (or injury) on a body, wherein a first sound pattern guides the person to pivot or rotate the phone in a first direction and a second sound pattern guides the person to pivot or rotate the phone in a second direction. In an example, AI (e.g. machine learning and/or artificial intelligence) can be used to identify an arcuate path (e.g. trajectory and sequence of orientations) in 3D space along which a phone (or other imaging device) should be moved to record images of a pathology (or injury) on a body from different perspectives. In an example, visual guidance can indicate the direction in which a phone (or other imaging device) should be moved relative to its current location and/or orientation.

In another example, a method can comprise using AI (e.g. machine learning and/or artificial intelligence) to display roll, pitch, and yaw directional indicators (e.g. in augmented reality) to guide a person concerning how to move a phone (or other imaging device) to record in-focus images of a pathology (or injury) on a body from different perspectives. In an example, a method for guiding a person concerning how to move a phone (or other imaging device) to record images of a pathology (or injury) on a body on a body can comprise using machine learning and/or artificial intelligence to provide the person with real-time instructions for changing phone location, yaw, pitch, and roll. In another example, a screen display or augmented reality display can show yaw, roll, or pitch of a phone (or other imaging device) relative to (the center of) a pathology (or injury) on a body.

In an example, a method for guiding a person concerning how to move a phone (or other imaging device) to record images of a pathology (or injury) on a body, wherein this guidance comprises an iterative, real-time sequence of real-time instructions and movements. In an example, a method for guiding a person concerning how to move a phone (or other imaging device) to record images of a pathology (or injury) on a body, wherein this guidance comprises a plurality of incremental (and iterative) instruction-movement cycles. In an example, a person holding a phone (or other imaging device) can be guided concerning how to move the phone relative to a pathology (or injury) on a body in a near-real-time iterative manner comprising multiple feedback cycles, wherein each feedback cycle comprises a data processor receiving an image of the pathology, analyzing the images, and recommending an action (e.g. moving the phone in a selected direction) for the person holding the phone, wherein each cycle takes less than 20 seconds.

In an example, a method for guiding a person concerning how to move a phone (or other imaging device) to record images of a pathology (or injury) on a body can include real-time visual guidance on the display of augmented reality eyewear, wherein this visual guidance comprises directional arrows, vectors, or other visual directional cues. In an example, the location of phone (or other imaging device) in 3D space can be determined by artificial intelligence analysis of images from: the phone (or other imaging device); and data from a motion sensor on the phone (or other imaging device).

In another example, a method can provide real-time guidance to a person moving a phone (or other imaging device), wherein this guidance helps the person to move the phone along a specified arcuate path (or shape) in space relative to a pathology (or injury) on a body, and wherein recording images of the pathology from multiple locations along the path enables creation of a digital 3D model of the pathology for medical evaluation. In an example, a method for guiding a person concerning how to move a phone (or other imaging device) to record images of a pathology (or injury) on a body can comprise a video (e.g. created by machine learning and/or artificial intelligence) which shows an image of the pathology and a virtual representation of the phone moving in a path (or shape) in 3D space over, across, and/or around the pathology.

In another example, a method, device, and/or system for recording images of a pathology or injury (e.g. a wound, ulcer, or skin lesion) on a portion of a body can comprise using machine learning and/or artificial intelligence to move a phone (or other imaging device), wherein the phone is moved along a virtual arcuate path (or shape) in space to record images of the pathology or injury from different perspectives. In an example, a phone (or other imaging device) can be moved along a selected path (or shape) in 3D space to record multiple images of a pathology (or injury) on a body from different angles, wherein these multiple images are integrated into a digital 3D model of the pathology which can be viewed from different angles.

In an example, guidance concerning how to move a phone (or other imaging device) to record a set of images of a pathology (or injury) can include instructions to move the phone in a circular and/or elliptical path (or shape) in (three-dimensional) space to record images of the pathology (or injury) from different angles. In an example, sequential images of a pathology (or injury) on a body recorded by a moving phone (or other imaging device) can be analyzed to determine whether the phone is moving along a desired path or shape in space. In an example, a method can comprise providing auditory, visual, and/or tactile guidance to a person concerning how to move a phone (or other imaging device) in an are over, across, and/or around a pathology (or injury) on a body.

In an example, a method can guide a person to move a phone (or other imaging device) along a concave path (or shape) in 3D space over, across, and/or around a pathology (or injury) on a body, wherein the shape of the path is a section of a sphere or a section of an ellipsoid, and wherein the pathology is under the concavity of the path. In an example, guidance concerning how to move a phone (or other imaging device) to record a set of images of a pathology (or injury) can include instructions to move the phone in a concave path (or shape) in three-dimensional space to record images of the pathology (or injury) from different angles, wherein the pathology is within (or at least below) the concavity of this path.

In another example, a method for guiding a person concerning how to move a phone (or other imaging device) over, across, and/or around a pathology (or injury) on a body can comprise creating an augmented reality display which shows the pathology, the phone, and a virtual arcuate shape in 3D space (e.g. section of a sphere or ellipsoid) along which the phone should be moved to record images of the pathology from multiple angles to create a digital 3D model of the pathology. In an example, a method for guiding a person concerning how to move a phone (or other imaging device) over, across, and/or around pathology (or injury) on a body can comprise: receiving images of the pathology which have been recorded by the phone; using machine learning and/or artificial intelligence to specify an arcuate 3D shape (e.g. a section of a sphere or ellipsoid) along and/or around which the phone should be moved to record images of the pathology from different perspectives (e.g. to create a digital 3D model of the pathology); and providing guidance (e.g. auditory guidance, visual guidance, and/or tactile guidance) to the person concerning how to move the phone along the arcuate 3D shape.

In another example, a method for guiding a person concerning how to move a phone (or other imaging device) over, across, and/or around a pathology (or injury) on a body can comprise creating an augmented reality image shown to the person, wherein the augmented reality image includes: the pathology; the phone; and a virtual arcuate surface in 3D space (e.g. a section of a sphere or ellipsoid) over, across, and/or around the pathology; wherein the person is guided (e.g. with auditory, visual, or tactile guidance) concerning how to move the phone along the virtual arcuate surface in order to record images of the pathology from different perspectives (e.g. to create a digital 3D model of the pathology). In an example, a method for guiding a person concerning how to move of a phone (or other imaging device) to record images of a pathology (or injury) can comprise instructing the person to move the phone along an arcuate path (e.g. a section of a circle or ellipse) or over an arcuate 3D shape (e.g. a section of a sphere or ellipsoid) which is over, across, and/or around the pathology.

In an example, a method for guiding a person concerning how to move a phone (or other imaging device) to record images of a pathology (or injury) on a body can comprise instructing the person to move the phone in a plane which is substantially parallel to a flat surface of a pathology or which is tangential to a curved surface of a pathology. In an example, a method for record images of a pathology (or injury) on a portion of a body can comprise guiding a person concerning how to move the phone (or other imaging device) along the surface of an virtual arcuate path (or shape) in space over, across, and/or around the pathology, wherein the plane of the phone is kept substantially tangential to the surface of the virtual arcuate path (or shape).

In an example, a method, device, or system can provide guidance to a person comprising (auditory, visual, or tactile) instructions concerning how to move a phone (or other imaging device) so that the changing plane of the phone remains substantially orthogonal to a radial vector extending out from (the center of) a pathology (or injury) on a body. In another example, the movement path of a phone (or other imaging device) in 3D space over, across, and/or around a pathology (or injury) on a body can be within a flat plane which is parallel or tangential to the surface of the pathology.

In another example, a method for guiding a person concerning how to move a phone (or other imaging device) over, across, and/or around a pathology (or injury) on a body can comprise: creating an augmented reality display which shows the pathology, the phone, and a virtual arcuate shape in 3D space (e.g. section of a sphere or ellipsoid) along which the phone should be moved to record images of the pathology on a body from multiple angles to create a digital 3D model of the pathology on a body; and guiding the person concerning how to move the phone in a back-and-forth, oscillating, serpentine, and/or spiral manner along this virtual arcuate shape in 3D space. In an example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has an undulating and/or serpentine shape.

In an example, the distances between arcs of a spiral and/or helical path along which a phone (or other imaging device) is moved to record images of a pathology (or injury) can vary with distance from the pathology. In an example, the distances between arcs of a spiral and/or helical path along which a phone (or other imaging device) is moved to record images of a pathology (or injury) can decrease with distance from the pathology. In an example, the distances between arcs of a spiral and/or helical path along which a phone (or other imaging device) is moved to record images of a pathology (or injury) can decrease with distance from the pathology so that there is a greater number (or higher density) of images as the phone approaches the apex of a virtual concave shape over, across, or around the pathology.

In another example, a method for guiding a person concerning how to move a phone (or other imaging device) to record images of a pathology (or injury) on a body can comprise guiding the person to move the phone back and forth over the surface of a virtual concave shape (e.g. a section of a sphere or ellipsoid). In an example, a method for guiding a person concerning how to move a phone (or other imaging device) to record images of a pathology (or injury) on a body can comprise guiding the person to move the phone back and forth (in a non-overlapping manner) over the surface of a virtual concave shape (e.g. a section of a sphere or ellipsoid) until the phone has recorded images of the pathology from substantially all areas, portions, or sectors of the concave shape.

In an example, a method for recording phone (or other imaging device) images of a pathology (or injury) on a body for medical evaluation can comprise guiding a person concerning how to move a phone along one or more virtual circular, semi-circular, spiral, or helical paths (or shapes) in space so as to record in-focus images of the pathology from different angles and distances. In an example, a method, device, and/or system for guiding a person concerning how to move a phone (or other imaging device) to record images of a pathology or injury (e.g. a wound or ulcer) on a portion (e.g. a foot) of a body can comprise a phone (or other imaging device), wherein the phone is configured to be moved along a virtual arcuate (e.g. undulating and/or starburst-shaped) path in space to record images of a pathology from different perspectives.

In an example, a path along which the phone is moved can be an undulating and/or starburst-shaped path along the surface of a section of a sphere (e.g. a hemisphere), wherein this section is centered on a pathology. In an example, guidance concerning how to move a phone (or other imaging device) to record a set of images of a pathology (or injury) can include instructions to move the phone in an oscillating (e.g. back and forth) path (or shape) in (three-dimensional) space to record images of the pathology (or injury) from different angles.

In an example, a method can guide a person concerning how to move a phone (or other imaging device) along a path (or shape) in 3D space in order to record in-focus images of a pathology (or injury) on a body from different angles, wherein this guidance comprises emission of light or display of graphical objects with an intensity that varies (e.g. decreases) with the distance of the phone from the path, thereby helping the person to move and keep the phone along the path. In an example, a screen display or augmented reality display can show the distance between a phone (or other imaging device) and a virtual path (or shape) across, over, or around a pathology (or injury) on a body. In an example, a method for guiding a person concerning how to move a phone (or other imaging device) to record images of a pathology (or injury) on a body can use sound, wherein the frequency, pitch, volume, or pulse rate of sound emitted from the phone changes if the phone deviates from a specified path (or shape) in space over, across, and/or around the pathology, and wherein the changes vary according to the direction and/or distance by which the phone deviates from the path.

In another example, a method for helping a person to record high-quality images of a pathology (or injury) on a body for medical evaluation using a phone (or other imaging device) can include providing the person with auditory guidance concerning how to move the phone along a specified arcuate path (or shape) in 3D space over, across, and/or around the pathology; wherein this auditory guidance comprises generation of sounds whose pitch, tone, frequency, and/or pulse rate changes when the phone deviates from the path. In an example, a method for helping a person to record high-quality images of a pathology (or injury) on a body for medical evaluation using a phone (or other imaging device) can include providing the person with tactile guidance concerning how to move the phone along a specified arcuate path (or shape) in 3D space over, across, and/or around the pathology; wherein this tactile guidance comprises changes in the magnitude, frequency, or pulse rate of vibrations when the phone deviates from the path.

In another example, an augmented reality display can provide guidance which helps a person to move a phone (or other imaging device) along a virtual path (or shape) in 3D space record images of a pathology (or injury) on a body, wherein the augmented reality display shows the virtual path (or shape) relative to the pathology, and wherein the virtual path is shown with a first display characteristic (e.g. a first color, thickness, brightness, or opacity level) when the phone is on the path and a second display characteristic (e.g. a second color, thickness, brightness, or opacity level) when the phone deviates from the path.

In an example, in an auditory guidance system, one or more characteristics (e.g. pitch, tone, volume, sequence, or pulse rate) of an emitted sound can be based on the direction and/or the amount of deviation of the location of a phone (or other imaging device) from a desired virtual path or shape in 3D space. In an example, sound emitted by a phone (or other imaging device) can have a higher pitch when the phone is on the correct path to record images of a pathology and can have a lower pitch when the phone deviates from the correct path. In an example, sound emitted by a phone (or other imaging device) can be louder when the phone is on the correct path to record images of a pathology and can be quieter (or even silent) when the phone deviates from the correct path.

In an example, a method can comprise providing real-time audio feedback (e.g. speech or tones) to help a person to keep phone (or other imaging device) moving along an identified path or shape in 3D space. In an example, a method can guide a person concerning how to move a phone (or other imaging device) along a path (or shape) in 3D space in order to record in-focus images of a pathology (or injury) on a body from different angles, wherein this guidance comprises a series of tones, sounds, and/or beeps. In an example, a method for guiding a person concerning how to move a phone (or other imaging device) to record images of a pathology (or injury) on a body on a body can comprise verbal cues (e.g. spoken words), wherein the verbal cues (e.g. spoken words) tell the person which direction to move the phone, and wherein the verbal cues are generated by real-time analysis of images from the phone by machine learning and/or artificial intelligence.

In an example, a method for guiding a person concerning how to move a phone (or other imaging device) to record images of a pathology (or injury) on a body on a body for medical evaluation can comprise providing verbal guidance including one or more phrases selected from the group comprising: “move the phone to the right”, “move the phone to the left”, “take it back now yall”, “one hop this time”, “two hops this time”, “right foot”, “let's stomp”, “left foot”, “let's stomp”, “cha now yall”, “turn it up”, and “now wave that fan”. In an example, a method for guiding a person concerning how to move a phone (or other imaging device) to record images of a pathology (or injury) on a body can include spoken instructions (e.g. directions in which the phone should be moved) emitted from the phone, smart eyewear, or another device.

In another example, a method of a method, device, and/or system for guiding a person concerning how to move a phone (or other imaging device) to record images of a pathology or injury (e.g. a wound, ulcer, or skin lesion) on a portion (e.g. foot) of a body can comprise: a phone (or other imaging device); wherein the phone is moved along a virtual arcuate path (or shape) in space to record images of the pathology or injury (e.g. wound, ulcer, or skin lesion) on a portion (e.g. foot) of the body from different perspectives; a first type (e.g. first pitch, frequency, level, or tone sequence) of sound which indicates that the phone is not on the path; and a second type (e.g. second pitch, frequency, level, or tone sequence) of sound which indicates that the phone is on the path. In an example, a person can be provided with auditory guidance (e.g. different emitted sounds or spoken words) concerning how to move a phone along a virtual arcuate path to record images of a pathology on a body. In another example, auditory guidance can comprise voice-based (e.g. spoken) instructions to a person moving a phone to record images of a pathology (or injury) on a body.

In an example, a method can comprise providing auditory (e.g. spoken words or sound tones), visual (e.g. written words or graphically-displayed objects), and/or tactile (e.g. vibrations) guidance to a person concerning how to move a phone (or other imaging device) in a virtual path (or shape) in 3D space over, across, and/or around a pathology (or injury) on a body. In another example, a method can comprise providing guidance to a person concerning how to move a phone (or other imaging device) to record images of a pathology (or injury) on a body; wherein the guidance is visual; and wherein the guidance further comprises displaying a first gauge to show the person when (or how) change the roll the phone, displaying a second gauge to show the person when (or how) to change the pitch of the phone, and/or displaying a third gauge to show the person when (or how) change the yaw of the phone.

In an example, a method can comprise: giving a person one or more auditory, visual, and/or tactile cues, signals, and/or communications with first characteristics if a phone (or other imaging device) is on a specified virtual path or shape to record images of a pathology (or injury); and giving the person one or more auditory, visual, and/or tactile cues, signals, and/or communications with second characteristics if the phone is not on the virtual path or shape. In an example, a method can use machine learning and/or artificial intelligence to identify and display an arcuate path (or shape) in 3D space along which a phone (or other imaging device) should travel in order to record images of a pathology (or injury) on a body. In an example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has a starburst and/or sunburst shape.

In another example, a method for guiding a person concerning how to move a phone (or other imaging device) to record images of a pathology (or injury) on a body on a body can comprise using machine learning and/or artificial intelligence to display directional graphics (e.g. arrows or vectors) which show the person how to change the location, yaw, pitch, and/or roll of the phone. In an example, a person can be provided with visual guidance (e.g. displayed directional indicators or words) concerning how to move a phone along a virtual arcuate path to record images of a pathology on a body. In an example, a visual display to guide a person concerning how to move a phone (or other imaging device) can comprise cross hairs displayed on a computer screen, wherein the cross hairs are aligned when the phone is in the correct location (and/or on the correct path) in 3D space.

In an example, a method can guide a person concerning how to move (or otherwise operate) a phone (or other imaging device) to record images of a pathology (or injury) on a body via an augmented reality display. In another example, a method for guiding a person concerning how to move a phone (or other imaging device) relative to a pathology (or injury) on a body can use the juxtaposition of a real object and a virtual object in an augmented reality display to guide the person concerning how to move the phone, wherein the person is instructed to move the phone until the real object and the virtual object are aligned in the augmented reality display.

In an example, guidance concerning how to move the phone (or other imaging device) to record images of a pathology (e.g. wound, ulcer, or skin lesion) on a body can be visual (e.g. directional indicators or words shown on a screen and/or in augmented reality). In an example, visual guidance for how to move a phone (or other imaging device) can comprise displaying one or more virtual objects near a pathology (or injury) on a body in an augmented reality display, wherein the one or more virtual objects include directional indicators (e.g. arrows) indicating how the phone should be moved in 3D space to record images of the pathology from different locations and/or angles.

In an example, a method can include providing a person with guidance concerning how to move a phone (or other imaging device) relative to a pathology (or injury) on a body in order to record useful images of the pathology for medical evaluation, wherein this guidance comprises aligning a first virtual object (or pattern) which is stationary relative to the pathology with a second virtual object (or pattern) which moves when the phone is moved, wherein the first and second virtual objects (or patterns) appear, along with the pathology, in an augmented reality display. In another example, a method for guiding a person concerning how to move a phone (or other imaging device) to record images of a pathology (or injury) on a body can comprise an augmented reality display which shows the pathology and a virtual representation of the phone moving in a path (or shape) in 3D space over, across, and/or around the pathology.

In an example, a method for guiding a person concerning how to move a phone (or other imaging device) to record images of a pathology (or injury) on a body can comprise providing a person with an augmented reality display which shows a virtual object (e.g. pointer, cursor, target symbol, cross hairs, or other geometric pattern) relative to the pathology, wherein the person is instructed to move the phone so as to align the virtual object with the pathology in the augmented reality display. In an example, an augmented reality display can show: a pathology (or injury) on a body; a current location and past path in 3D space of a phone (or other imaging device); and an identified virtual path or shape in 3D space along which the phone should be moved to record images of the pathology.

In an example, an augmented reality display can show a pathology (or injury) on a body, the location of a phone (or other imaging device), and how the phone should be moved to record images of the pathology. In another example, guidance concerning how to move a phone (or other imaging device) to record a set of images of a pathology (or injury) can comprise a suggested phone movement path (or shape) in three-dimensional space which is superimposed on an image of the pathology (or injury) in an augmented reality display. In an example, visual guidance for how to move a phone (or other imaging device) can comprise the display of one or more virtual objects relative to a pathology (or injury) on a body in an augmented reality display.

In an example, a method can comprise: (a) receiving a first set of one or more images of a pathology (or injury) on a body which have been recorded by a phone (or other imaging device); (b) analyzing the first set of images using machine learning and/or artificial intelligence to identify an arcuate path (or shape) in 3D space along which the phone should be moved to record a superior second set of one or more images of the pathology; and (c) generating tactile directional indicators (e.g. vibration sequences) which guide a person concerning move the phone along the identified path.

In an example, a method for guiding a person concerning how to move a phone (or other imaging device) to record images of a pathology (or injury) on a body can include using vibration of the phone to guide the person concerning how to move the phone relative to the pathology. In another example, a method to guide a person concerning how to move a phone (or other imaging device) to record images of a pathology (or injury) on a body can comprise providing vibrations or vibration sequences which guide a person concerning how to move a phone (or other imaging device), wherein (changes in) the frequency and/or amplitude of the vibrations guides the person concerning whether to move the phone closer to (or farther from) the pathology.

In an example, a method to guide a person concerning how to move a phone (or other imaging device) to record images of a pathology (or injury) on a body can comprise providing vibrations or vibration sequences via a handheld or wearable device which guide the person concerning how to move the phone (or other imaging device). In another example, a method, device, or system can provide a person with real-time AI-generated tactile guidance concerning how to move a phone (or other imaging device) along a specified arcuate path (or shape) in 3D space over, across, and/or around a pathology (or injury) on a body.

In an example, a system for helping a person to record high-quality images of a pathology (or injury) on a body for medical evaluation using a phone (or other imaging device) can comprise: a phone; a camera on the phone; a data processor; and a vibrating component; wherein the data processor receives a first set of images of a pathology which has been recorded by the camera; wherein the data processor (e.g. using machine learning and/or artificial intelligence) analyzes the first set of images to determine an arcuate path (or shape) in 3D space along which the phone should be moved to record a superior second set of images of the pathology; and wherein the system uses the vibrating component to provide a person with real-time tactile guidance (e.g. vibration sequences) concerning how to move the phone along the arcuate path in 3D space. In another example, a method can include providing guidance to a person concerning how to move a phone (or other imaging device) relative to a pathology (or injury) on a body in order to record useful images of the pathology for medical evaluation, wherein this guidance is in the form of words which are displayed on the phone and/or a secondary device (e.g. smart eyewear, a smart watch, or a second phone).

In an example, an augmented reality display on a second phone to guide a person concerning how to move a first phone to record images of a pathology (or injury) can show one or more of the following views: a current view of the first phone; a current view of the pathology; a view of a path (e.g. a virtual arcuate path or shape in 3D space) along which the first phone should be moved to record images of the pathology from different perspectives; an oblique side view of the first phone, the pathology, and the virtual path; an overhead (e.g. top down) view of the first phone, the pathology, and the virtual path; a view of the pathology from the perspective of the first phone (e.g. from the camera on the first phone); one or more directional indicators which show which direction the first phone should be moved now (e.g. relative to the path and/or the pathology); one or more directional indicators which show how the first phone should be rotated or pivoted now (e.g. relative to the path and/or the pathology); an animation which shows the first phone moving along the virtual path; and a view of which portions of the path have been traveled by the first phone and which portions of the path have not yet been traveled by the first phone.

In an example, guidance concerning how to move a phone (or other imaging device) to record a set of images can comprise a virtual geometric pattern (e.g. a target or cross-hairs pattern) shown relative to the pathology (or injury) on a body in an augmented reality display via a secondary device (such as a second phone, a smart watch, or smart eyewear). In an example, visual guidance for moving a first phone can be shown on a secondary phone. In an example, a method can comprise: (a) using augmented reality (e.g. via a phone screen or an eyewear display) which shows a pathology (or injury) on a body; a phone (or other imaging device) near the pathology; a virtual path (or shape) in 3D space over, across, and/or around the pathology; and a virtual object on the virtual path; and (b) guiding a person to move the phone in alignment with the virtual object as the object traces (e.g. moves along) the virtual path in the augmented reality display.

In an example, a method for guiding a person concerning how to move a phone (or other imaging device) to record images of a pathology (or injury) on a body on a body can include a display (e.g. a screen on the phone or display via augmented reality eyewear) which shows: the pathology; the phone; and how the phone should be moved relative to the pathology. In an example, guidance concerning how to move a phone (or other imaging device) to record a set of images can comprise a virtual geometric pattern (e.g. a target or cross-hairs pattern) shown relative to the pathology (or injury) on a body in an augmented reality display (e.g. via a phone screen or smart eyewear). In another example, a method for guiding a person to record images of a pathology (or injury) on a body can comprise: receiving images of a pathology (or injury) on a body recorded by a phone (or other imaging device); and providing visual guidance to a person concerning how to move the phone, wherein this guidance is provided via smart eyewear (e.g. augmented reality eyewear).

In an example, an augmented reality display in smart eyewear to guide a person concerning how to move a phone to record images of a pathology (or injury) can show one or more of the following views: a current view of the phone; a current view of the pathology; a view of a path (e.g. a virtual arcuate path or shape in 3D space) along which the phone should be moved to record images of the pathology from different perspectives; an oblique side view of the phone, the pathology, and the virtual path; an overhead (e.g. top down) view of the phone, the pathology, and the virtual path; a view of the pathology from the perspective of the phone (e.g. from the camera on the phone); one or more directional indicators which show which direction the phone should be moved now (e.g. relative to the path and/or the pathology); one or more directional indicators which show how the phone should be rotated or pivoted now (e.g. relative to the path and/or the pathology); an animation which shows the phone moving along the virtual path; and a view of which portions of the path have been traveled by the phone and which portions of the path have not yet been traveled by the phone.

In another example, visual guidance for how to move a phone (or other imaging device) can comprise the display of one or more virtual objects relative to a pathology (or injury) on a body in an augmented reality display, wherein this display is shown via augmented reality eyewear. In an example, a method for guiding a person concerning how to move a smart watch (or watch band) with a camera to record images of a pathology (or injury) on a body can comprise providing the person with auditory guidance (e.g. sounds or sound sequences) which indicate whether the person should move the watch closer to the pathology or farther from the pathology.

In an example, an augmented reality display in smart eyewear to guide a person concerning how to move a smart watch (with a camera) to record images of a pathology (or injury) can show one or more of the following views: a current view of the smart watch; a current view of the pathology; a view of a path (e.g. a virtual arcuate path or shape in 3D space) along which the smart watch should be moved to record images of the pathology from different perspectives; an oblique side view of the smart watch, the pathology, and the virtual path; an overhead (e.g. top down) view of the smart watch, the pathology, and the virtual path; a view of the pathology from the perspective of the smart watch (e.g. from the camera on the smart watch); one or more directional indicators which show which direction the smart watch should be moved now (e.g. relative to the path and/or the pathology); one or more directional indicators which show how the smart watch should be rotated or pivoted now (e.g. relative to the path and/or the pathology); an animation which shows the smart watch moving along the virtual path; and a view of which portions of the path have been traveled by the smart watch and which portions of the path have not yet been traveled by the smart watch.

In an example, a method can include providing a person with guidance concerning how to move a phone (or other imaging device) to record images of a pathology (or injury) on a body for medical evaluation, wherein this guidance is in the form of a suggested phone movement path (or shape) in 3D space which is shown (e.g. in augmented reality) via a phone, a smart watch, or smart eyewear. In an example, a mobile imaging device which is used to record images of a pathology (or injury) on a body can be selected from the group consisting of: smart phone; other communication device; augmented reality eyewear; smart watch; watch band; wrist band or bracelet; and smart finger ring. In an example, a second set of images of a pathology (e.g. wound, ulcer, or skin lesion) on a body can be used to create a digital 3D model of the pathology. In an example, images of a pathology (or injury) on a body which are recorded from multiple perspectives can be integrated (e.g. by machine learning and/or artificial intelligence) into a digital 3D model of the pathology for diagnostic and/or therapeutic purposes.

In another example, a machine learning and/or artificial intelligence model can be trained on prior anatomical images of a person being imaged in addition to anatomical images of a large number of other people. In an example, a method for guiding a person concerning how to move a phone (or other imaging device) to record images of a pathology (or injury) on a body can comprise instructing the person to align a perimeter (or outline) of a pathology (or injury) with a previously-documented perimeter of the pathology (or injury) in an augmented reality display. In another example, machine learning and/or artificial intelligence can align images of a pathology (or injury) on a body which are recorded at different times (e.g. align the centers or outlines of the pathology recorded at different times) in order to measure, display, and/or evaluate changes in the pathology.

In an example, a method can comprise recording an image of an object with a known color spectrum which is next to a pathology (or injury) on a body, wherein the known color spectrum is used to calibrate the colors of the of the pathology in the image. In an example, a method can use machine learning and/or artificial intelligence to adjust (e.g. calibrate) the color spectrum of images recorded by a phone (or other imaging device) based (at least partially) on the type and/or model of the phone, wherein the type and/or model is reported by a person using the phone or automatically received from the phone.

In an example, a method for guiding a person concerning how to use a phone (or other imaging device) to record images of a pathology (or injury) on a body can comprise instructing the person to enter the type and/or model of the phone into a user interface. In an example, a method, device, or system can use machine learning and/or artificial intelligence to guide a person concerning how to record a second (guided) set of images of a pathology (or injury) on a body which are superior to those recorded in a first (unguided) set of images because the second set includes an environmental object near the pathology for calibration of color and/or size.

In another example, a method for guiding a person concerning how to use a phone (or other imaging device) to record images of a pathology (or injury) on a body can comprise instructing the person to change the level and/or type of ambient light. In an example, a method for recording images of a pathology (or injury) on a body can comprise: receiving a first image of the pathology; analyzing the image to evaluate illumination level in the image; and remotely activating a light on the phone if needed for a second image of the pathology.

In an example, a method can ask person to adjust ambient lighting for recording images by: activating a lighting component (e.g. flash or flashlight) on a phone (or other imaging device); by removing or adding ambient lighting sources; or by moving to different location in the environment. In an example, a method for providing guidance concerning how to use a phone (or other imaging device) relative to a pathology (or injury) on a body can comprise analyzing a first set of images of the pathology to evaluate ambient lighting and guiding the person concerning to moving to a different location with different ambient lighting and/or to changing local light-emitting sources.

In another example, a method can comprise: receiving a first set of images of a pathology (or injury) on a body which have been recorded by a phone (or other imaging device); using machine learning and/or artificial intelligence to identify problems with the first set of images which make them unsatisfactory for use in medical evaluation; and using machine learning and/or artificial intelligence to remotely change phone settings and/or actions before a second set of images of the pathology are recorded; wherein the settings and/or actions are selected from the group consisting of: activating a light emitter on the phone; changing illumination level; changing the angle and/or orientation of the phone camera; changing the focal distance of the phone camera; changing the color filter of the phone camera; and changing the zoom or magnification setting of the phone camera.

In an example, a method can include using machine learning and/or artificial intelligence to remotely control one or more functions of a phone (or other imaging device) to improve the quality of images of a pathology (or injury) on a body which are recorded by the phone, wherein these functions are selected from the group consisting of: changing color filter; changing focal distance; changing focal vector and/or angle; changing focal width and/or scope; changing focal zoom; changing frame rate; changing illumination intensity; changing illumination wavelength and/or spectral distribution; changing imaging aperture; changing magnification level; changing shutter speed; remotely activating image (still or video) recording; and remotely ending image (still or video) recording.

In an example, a method can include: using machine learning and/or artificial intelligence to (automatically and/or remotely) activate a phone (or other imaging device) to record images of a pathology (or injury) on a body when the phone is at a specified location and/or moving along a specified path (or shape) in 3D space. In an example, a method for guiding a person concerning how to record phone (or other imaging device) images of a pathology (or injury) on a body for medical evaluation can include giving a remote data processor (e.g. a remote device or server) remote control over one or more of the phone's functions selected from the group consisting of: phone camera focal distance; phone camera image magnification level; phone camera spectral filter setting; phone flashlight (e.g. light emitter) function; starting phone camera image video recording; ending phone camera image video recording; and recoding a still image via phone camera.

In another example, a method for recording phone (or other imaging device) images of a pathology (or injury) on a body for medical evaluation can include giving a remote data processor (e.g. a remote device or server) remote control over one or more of the phone's camera functions. In an example, a method, device, and/or system for recording images of a pathology or injury (e.g. a wound, ulcer, or skin lesion) on a portion of a body can comprise using machine learning and/or artificial intelligence to remotely control a (home and/or personal care) robot to move a phone (or other imaging device), wherein the robot moves the phone along a virtual arcuate path (or shape) in space to record images of the pathology or injury from different perspectives.

In an example, a first person can have a pathology (or injury) on their body and a second person can move a phone (or other imaging device) over, across, and/or around the pathology to record images for medical evaluation. In an example, a person can move a phone (or other imaging device) to record images of a pathology (or injury) on their own body for medical evaluation. In an example, a pathology (or injury) can be a pressure ulcer. In an example, a pathology (or injury) can be a wound, a skin lesion, and/or another type of skin abnormality. In another example, a pathology can be a skin wound. In an example, a pathology may potentially be skin cancer. In another example, an injury can be a skin laceration.

In an example, analysis of phone (or other imaging device) images of a pathology (or injury) on a body can include evaluation of the size, area, depth, volume, shape, outline, texture, color, temperature, spectral absorption distribution, and/or movement of the pathology. In another example, a method can comprise using machine learning and/or artificial intelligence to analyze phone (or other imaging device) images of a pathology (or injury) on a body for teledermatology applications. In an example, the location of phone (or other imaging device) in 3D space can be determined by artificial intelligence analysis of images from the phone (or other imaging device).

In an example, one or more of the following methods can be used to analyze phone (or other imaging device) images of a pathology (or injury) on a body and/or to guide a person holding the phone concerning how to move the phone to record images: artificial intelligence, Bayesian analysis, data analytics, deep learning algorithms, Fourier transformation, fuzzy logic, inductive logic programming, least squares estimation, linear discriminant analysis, logistic discrimination, machine learning, multivariate analysis, multivariate linear regression, pattern recognition, principle components analysis, random forest analysis, and time-series analysis.

In an example, a method can comprise: receiving a first set of images of a pathology (or injury) on a body recorded by a phone (or other imaging device), analyzing the first set of images to identify an arcuate path (or shape) in 3D space along which the phone should be moved to record a superior second set of images of the pathology; receiving data from a motion sensor on the phone; and providing (auditory, visual, or tactile) guidance to the person concerning how the phone should be moved to stay on the path based on (real time) analysis of data from the motion sensor and images from the phone. In an example, a method can guide a person concerning how to record a second set of images of a pathology (or injury) on a body based on analysis of a first set of images of the pathology (e.g. to correct problems with the first set), wherein the second set of images are more accurate for medical evaluation purposes than the first set.

In an example, a method for guiding a person concerning how to move a phone (or other imaging device) to record images of a pathology (or injury) on a body can comprise: receiving a first set of images of the pathology recorded by the phone; using machine learning and/or artificial intelligence to analyze the first set of images to identify an arcuate path (or shape) in 3D space along which the phone should be moved to record a second set of images of the pathology; and guiding the person (e.g. with visual, auditory, or tactile guidance) concerning how to move the phone along this path.

In another example, a method for recording images of a pathology (or injury) on a body can comprise: (a) receiving a first set of one or more images of a pathology (or injury) on a body which have been recorded by a phone (or other imaging device); (b) using machine learning and/or artificial intelligence to analyze the first set of one or more images and to specify a virtual arcuate path (or shape) in space from which a second set of one or more images of the pathology should be recorded; and (c) guiding a person concerning how to move the phone along the virtual arcuate path in space to record the second set of one or more images.

In an example, a method to guide a person concerning how to move a phone (or other imaging device) to record images of a pathology (or injury) on a body can comprise: asking a person to use a phone to record a first set of one or more images of the pathology; receiving the first set of one or more images from the phone; analyzing the first set of one or more images using machine learning and/or artificial intelligence; determining a virtual path or shape in 3D space along which the phone should be moved to record a second set of one or more images which is better focused, more accurate, more comprehensive, and/or more useful for medical evaluation than the first set; and providing guidance to the person concerning how to move the phone along this virtual path or shape.

In another example, a method to guide a person concerning how to move a phone (or other imaging device) to record images of a pathology (or injury) on a body can comprise: receiving a first set of one or more images of a pathology on a body recorded by the phone (or other imaging device); analyzing the first set of one or more images; and guiding the person concerning how to move the phone to record a second set of one or more images of the pathology. In an example, a second set of one or more images of a pathology (e.g. wound, ulcer, or skin lesion) on a body can be superior for medical evaluation purposes than a first set of one or more images of the pathology.

In an example, an AI-guided second set of one or more images of a pathology (or injury) on a body can enable more accurate measurement, characterization, and/or medical evaluation of a pathology than an unguided first set of one or more images. In an example, guidance concerning how to move a phone (or other imaging device) relative to a pathology (or injury) on a body to record a second set of guided images of the pathology can be at least partly based on analysis of a first set of unguided images of the pathology.

In an example, a method can comprise: providing a person with a first level of guidance concerning how to move and/or otherwise operate a phone (or other imaging device) to record a first set of images of a pathology (or injury) on a body for medical diagnostic purposes; analyzing the first set of images (e.g. with machine learning and/or artificial intelligence) to evaluate their usefulness for medical diagnostic purposes; and providing the person with a second level of guidance concerning how to move and/or otherwise use the phone to record a second set of images of the pathology if the first set is not satisfactory for medical diagnostic purposes; wherein the second level of guidance differs from the first level of guidance in one or more ways selected from the group consisting of: different type of sensory modality (e.g. auditory, visual, and tactile) used to provide guidance; greater number of devices (e.g. phone, smart eyewear, smart watch, wrist band, smart ring) used to provide guidance; greater number of sensory modalities (e.g. auditory, visual, and tactile) used simultaneously to provide guidance; guidance with multiple pauses for confirmation of understanding; more detailed or specific guidance in the same modality; and slower-paced guidance.

In another example, a method, device, or system can use machine learning and/or artificial intelligence to guide a person concerning how to record a second (guided) set of images of a pathology (or injury) on a body which are superior to those recorded in a first (unguided) set of images because the second set shows the pathology in sharper focus. In an example, a method, device, or system can use machine learning and/or artificial intelligence to guide a person concerning how to record a second (guided) set of images of a pathology (or injury) on a body which are superior to those recorded in a first (unguided) set of images because the second set shows the pathology from a greater range of distances. In another example, an AI-guided second set of one or more images of an abnormality on a pathology (or injury) on a body can enable more accurate assessment of the size, depth, shape, texture, color, healing, and/or pathology of a pathology than an unguided first set of one or more images.

In an example, a method can include providing guidance and/or instruction to a person concerning how to move a phone (or other imaging device) to one or more different locations to record images of a pathology (or injury) on a body from different distances and/or different angles, wherein this guidance and/or instruction includes the directions in which the phone should be moved. In an example, a person with a pathology (or injury) on their body can receive guidance from a machine-learning or artificial intelligence system concerning how to move a phone (or other imaging device) over, across, and/or around the pathology to record images for medical evaluation. In an example, a method can comprise providing a person with guidance concerning how to move a phone (or other imaging device) so as to maintain focal direction toward the (center of) a pathology (or injury) on a body. In an example, a method for guiding a person concerning how to use a phone (or other imaging device) to record images of a pathology (or injury) on a body can comprise instructing the person to change the focal distance setting of the phone.

In an example, a method for providing guidance concerning how to move a phone (or other imaging device) relative to a pathology (or injury) on a body can comprise: using machine learning and/or artificial intelligence to evaluate in (close to) real time whether images recorded by the phone are in focus; and guiding a person (with auditory, visual, and/or tactile cues) concerning how to move the phone if the image is not in focus. In another example, a method for using a phone (or other imaging device) to record images of a pathology (or injury) on a body can include allowing a machine learning and/or artificial intelligence system to automatically adjust the focusing function of the phone to ensure that the pathology remains in focus as the phone is moved over, across, and/or around the pathology.

In an example, a method can comprise: giving a person one or more auditory, visual, and/or tactile cues, signals, and/or communications with first characteristics if a phone (or other imaging device) should be moved closer to a pathology (or injury) on a body; and giving the person one or more auditory, visual, and/or tactile cues, signals, and/or communications with second characteristics if the phone should be moved father from the pathology (or injury). In another example, a method for guiding a person concerning how to move a phone (or other imaging device) to record images of a pathology (or injury) on a body on a body can comprise verbal cues (e.g. spoken words) such as “please move phone a little closer to the body”, “please move the phone a little farther from the body”, “please move the phone a little to the right”, “please move the phone a little to the left”, and “please tilt the phone a bit to keep it pointed toward the body”.

In an example, a method for guiding a person concerning how to move a phone (or other imaging device) to record images of a pathology (or injury) on a body can comprise instructing the person to move the phone farther from (or closer to) the pathology. In an example, a method to guide a person concerning how to move a phone (or other imaging device) to record images of a pathology (or injury) on a body can comprise emission of light with a first color when the phone is too far from the pathology, emission of light with a second color when the phone is the proper distance from the pathology, and emission of light with a third color when the phone is too close to the pathology.

In an example, a method, device, or system can provide a person with real-time AI-generated auditory guidance concerning how to move a phone (or other imaging device) along a specified path (or shape) in 3D space over, across, and/or around a pathology (or injury) on a body, wherein a first sound pattern guides the person to move the phone closer to the pathology and a second sound pattern guides the person to move the phone farther from the pathology. In an example, guidance concerning how to move a phone (or other imaging device) to record a set of images of a pathology (or injury) can include instructions to change the distance between the pathology (or injury) and the phone.

In an example, sound emitted by a phone (or other imaging device) can have a faster pulse rate when the phone is too close to a pathology for accurate (e.g. in focus) imaging and can have a slower pulse rate when the phone is the correct distance from the pathology for accurate (e.g. in focus) imaging. In another example, sound emitted by a phone (or other imaging device) can have a lower pitch when the phone is too far from a pathology for accurate (e.g. in focus) imaging and can have a higher pitch when the phone is the correct distance from the pathology for accurate (e.g. in focus) imaging. In an example, sound emitted by a phone (or other imaging device) can have a slower pulse rate when the phone is too close to a pathology for accurate (e.g. in focus) imaging and can have a faster pulse rate when the phone is the correct distance from the pathology for accurate (e.g. in focus) imaging. In another example, sound emitted by a phone (or other imaging device) can be quieter (or even silent) when the phone is too far from a pathology for accurate (e.g. in focus) imaging and can be louder when the phone is the correct distance from the pathology for accurate (e.g. in focus) imaging.

In an example, a method can display an image which shows an arcuate path (or shape) in 3D space along which a phone (or other imaging device) should travel to record images of a pathology (or injury) on a body and also directional graphics (e.g. arrows or vectors) which show the orientations that the phone should have as it travels along this path. In an example, a method can include using data from a motion sensor in a phone (or other imaging device) to help track the position and/or orientation of the phone in space and to determine whether the phone is moving along a selected path (or shape) in space with the proper orientation to record images of a pathology (or injury) on a body. In an example, a method for guiding a person concerning how to move a phone (or other imaging device) to record images of a pathology (or injury) on a body can comprise using an augmented reality display to show visual cues (such as arrows, vectors, geometric patterns, or words) which guide the person concerning in which direction (or to which orientation) the phone should be moved.

In an example, a method for guiding a person concerning how to move a phone (or other imaging device) to record images of a pathology (or injury) on a body can comprise emitting light with different colors or color sequences which indicate that the phone should be moved in different directions or rotated to different orientations. In an example, a method for providing guidance concerning how to move a phone (or other imaging device) relative to a pathology (or injury) on a body can comprise guiding a person concerning how to pan, rotate, pivot, and/or tilt the phone to keep the phone's camera focal vector directed toward the pathology while the phone is moved on a virtual (arcuate) path or shape which is over, across, and/or around the pathology.

In another example, a method with machine-based and/or AI-based analysis of phone (or other imaging device) images of a pathology (or injury) on a body can include analysis of the angle between the phone and the pathology to determine whether the phone should be moved to the right or left and/or rotated in order to record images (e.g. to create a digital 3D model of the pathology). In an example, a method, device, or system can provide guidance to a person comprising (auditory, visual, or tactile) instructions concerning how to rotate a phone (or other imaging device) while it moves around a pathology (or injury) on a body so that the intersection angle between a plane of the phone and a radial vector extending out from (the center of) the pathology on a body is between 80 and 100 degrees. In another example, visual guidance can indicate the amount (e.g. rotational angle) by which a phone (or other imaging device) should be rotated and/or pivoted.

In an example, a method can comprise providing roll, pitch, and yaw directional indicators to a person concerning how to move a phone (or other imaging device) in order to records in-focus images of a pathology (or injury) on a body from different perspectives. In an example, a method can comprise using machine learning and/or artificial intelligence to: (a) identify a path (e.g. a trajectory or sequence of orientations) in 3D space along which a phone (or other imaging device) should be moved to record images of a pathology (or injury) on a body from different perspectives, wherein the path includes a changing location of the center (e.g. the centroid) of the phone and a changing orientation (e.g. roll, pitch, and yaw) of the phone; and (b) provide auditory, visual, or tactile guidance to a person concerning how to move the phone along this path.

In an example, a method for guiding a person concerning how to move a phone (or other imaging device) to record images of a pathology (or injury) on a body on a body can comprise using machine learning and/or artificial intelligence to display directional graphics (e.g. arrows or vectors) in augmented reality which show the person how to change the location, yaw, pitch, and/or roll of the phone. In an example, AI (e.g. machine learning and/or artificial intelligence) can be used to identify a path (or shape) (e.g. trajectory and sequence of orientations) in 3D space along which a phone (or other imaging device) should be moved to record images of a pathology (or injury) on a body from different perspectives, wherein the path includes both changing location of the center (e.g. the centroid) of the phone and the changing orientation (e.g. roll, pitch, and yaw) of the phone.

In an example, a method for guiding a person concerning how to move a phone (or other imaging device) to record images of a pathology (or injury) on a body can comprise providing auditory, visual, and/or tactile guidance concerning how the person move the phone; wherein the guidance comprises a series of incremental (e.g. short distance or short time) movements. In another example, a person holding a phone (or other imaging device) can be guided concerning how to move the phone relative to a pathology (or injury) on a body in a near-real-time iterative manner comprising multiple feedback cycles, wherein each feedback cycle comprises a data processor receiving an image of the pathology, analyzing the images, and recommending an action (e.g. moving the phone in a selected direction) for the person holding the phone. In an example, visual guidance can indicate the distance that a phone (or other imaging device) should be moved relative to its current location and/or orientation.

In another example, images of a pathology (or injury) on a body recorded by a phone (or other imaging device) held by a person can be analyzed by machine learning methods and/or artificial intelligence and guidance can be provided concerning how the person should move the phone in real-time, wherein real time means within seconds or (at most) a couple minutes. In an example, a method can include prompting and/or guiding a person to move a phone (or other imaging device) in a selected direction, to a selected location, and/or along a selected path (or shape) in 3D space in order to record images of a pathology (or injury) on a body. In an example, a method can show an arcuate path (or shape) in 3D space along which a phone (or other imaging device) should travel in order to record images of a pathology (or injury) on a body.

In an example, a method, device, and/or system for recording images of a pathology or injury (e.g. a wound, ulcer, or skin lesion) on a portion of a body can comprise moving a phone (or other imaging device), wherein the phone is moved along a virtual arcuate path (or shape) in space to record images of the pathology or injury from different perspectives. In an example, a method, device, and/or system for recording images of a pathology or injury (e.g. a wound, ulcer, or skin lesion) on a portion of a body can comprise using machine learning and/or artificial intelligence to guide a person concerning how to move a phone (or other imaging device), wherein the person moves the phone along a virtual arcuate path (or shape) in space to record images of the pathology or injury from different perspectives.

In an example, guidance concerning how to move a phone (or other imaging device) can comprise a virtual pathway or shape in three-dimensional space along which a phone should be moved to record images of a pathology (or injury) on a body from multiple distances and/or angles. In another example, method can provide continual, real-time guidance concerning how to move a phone along a path or shape in 3D space. In an example, shape of virtual path (or shape) along which a phone (or other imaging device) travels can be at least partly based on the shape (e.g. outline) of a pathology (or injury) on a body. In another example, a method can comprise using machine learning and/or artificial intelligence to provide (auditory, visual, or tactile) guidance to a person concerning how to move a phone (or other imaging device) in an are over, across, and/or around a pathology (or injury) on a body.

In an example, a method can guide a person to move a phone (or other imaging device) along a concave path (or shape) in 3D space over, across, and/or around a pathology (or injury) on a body, wherein the shape of the path is a section of a circle, a section of an ellipse, or another type of conic section, and wherein the pathology is under the concavity of the path. In an example, the movement path of a phone (or other imaging device) over, across, and/or around a pathology (or injury) on a body can be along the surface of a virtual concave 3D shape (e.g. a half sphere or half ellipsoid) in space which is centered on a location on (e.g. the centroid of) the pathology.

In an example, a method for guiding a person concerning how to move a phone (or other imaging device) over, across, and/or around a pathology (or injury) on a body can comprise: (a) showing the pathology in an augmented reality display (e.g. on a phone or eyewear); and (b) showing an arcuate 3D shape (e.g. a section of a sphere or ellipsoid) over, across, and/or around the pathology in the augmented reality display; wherein the phone should be moved along and/or over the arcuate 3D shape to record images of the pathology from different perspectives (e.g. to construct a digital 3D model of the pathology); wherein areas of the arcuate 3D shape where the phone has already been moved (e.g. from which images of the pathology have been recorded) are shown in a first manner (e.g. with a first color or transparency level); and wherein areas of the arcuate 3D shape where the phone has not yet been moved (e.g. from which images of the pathology have not yet been recorded) are shown in a second manner (e.g. with a second color or transparency level).

In another example, a method for guiding a person concerning how to move a phone (or other imaging device) over, across, and/or around pathology (or injury) on a body can comprise: receiving images of the pathology which have been recorded by the phone; using machine learning and/or artificial intelligence to specify an arcuate 3D shape (e.g. a section of a sphere or ellipsoid) along which the phone should be moved to record images of the pathology from different perspectives (e.g. to create a digital 3D model of the pathology); and guiding the person concerning how to move the phone along and/or around the arcuate 3D shape.

In an example, a method for guiding a person concerning how to use a phone (or other imaging device) to record images of a pathology (or injury) on a body for medical evaluation can comprise prompting to person to perform one or more of the following actions: activating a light emission (e.g. a flashlight) function on the phone or another device; aligning a perimeter of a pathology (or injury) with a previously-documented perimeter of the pathology (or injury) in an augmented reality display; aligning the pathology with a previous image of the pathology in an augmented reality display; changing the magnification setting on the phone; changing the focal distance setting on the phone; changing the light spectral filter on the phone; changing the zoom setting on the phone; changing the level and/or type of ambient light; entering the type and/or model of phone into a user interface; moving the phone along a virtual path in space which is shown on an augmented reality display; moving the phone away from the pathology; moving the phone closer to the pathology; moving the phone in a plane which is parallel to the surface of the pathology; moving the phone in a plane which is tangential to the surface of the pathology; moving the phone along an arcuate path (e.g. section of a circle or ellipse) over the pathology; moving the phone along an arcuate 3D shape (e.g. section of a sphere or ellipsoid) over the pathology; moving the phone to align a virtual object or pattern with a view of the pathology in an augmented reality display; moving the phone to align two virtual objects or patterns in an augmented reality display; moving the phone to the left; moving the phone to the right; moving to a different location with a different level and/or type of ambient light; pivoting or rotating the phone; and selecting an object with a known color spectrum and/or size and placing the object near the pathology.

In an example, a virtual path or shape along which a phone (or other imaging device) should be moved to record images of a pathology (or injury) can be a section of a circle, sphere, ellipse, or ellipsoid. In an example, a method for moving a phone (or other imaging device) to record images of a pathology (or injury) on a portion of a body can comprise moving the phone along the surface of an virtual arcuate path (or shape) in space over, across, and/or around the pathology, wherein the plane of the phone is kept substantially tangential to the surface of the virtual arcuate path (or shape).

In another example, a method for record images of a pathology (or injury) on a portion of a body can comprise using machine learning and/or artificial intelligence to guide a person concerning how to move the phone (or other imaging device) along the surface of an virtual arcuate path (or shape) in space over, across, and/or around the pathology, wherein the plane of the phone is kept substantially tangential to the surface of the virtual arcuate path (or shape). In an example, a method, device, or system can provide guidance to a person comprising (auditory, visual, or tactile) instructions concerning how to move a phone (or other imaging device) so that the changing plane of the phone remains with 10 degrees of being orthogonal to a radial vector extending out from (the center of) a pathology (or injury) on a body. In an example, a method for guiding a person concerning how to move a phone (or other imaging device) to record images of a pathology (or injury) on a body can comprise instructing the person to move the phone to the right, to the left, or right-and-left in an oscillating manner.

In an example, a method for guiding a person concerning how to move a phone (or other imaging device) to record images of a pathology (or injury) on a body can comprise an augmented reality display which shows a virtual object (e.g. pointer, cursor, target symbol, cross hairs, or other geometric pattern), a virtual path (or shape) in 3D space relative to the pathology, and the pathology, wherein the person is instructed to move the phone so as to move the virtual object back and forth along the virtual path. In another example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has a spiral and/or helical shape.

In an example, a method for guiding a person concerning how to move a phone (or other imaging device) to record images of a pathology (or injury) on a body can comprise guiding the person to move the phone back and forth over the surface of a virtual concave shape (e.g. a section of a sphere or ellipsoid) until the phone has recorded images of the pathology from substantially all portions (e.g. all locations) on the surface of the virtual concave shape. In another example, a method for guiding a person concerning how to move a phone (or other imaging device) to record images of a pathology (or injury) on a body can comprise guiding the person to move the phone back and forth (in a non-overlapping manner) over the surface of a virtual concave shape (e.g. a section of a sphere or ellipsoid) until the phone has recorded images of the pathology from substantially all latitudinal and longitudinal sectors of the concave shape.

In an example, a method for recording phone (or other imaging device) images of a pathology (or injury) on a body for medical evaluation can comprise guiding a person concerning how to move (e.g. rotate, pivot, and/or tilt) the phone to keep the plane of the phone substantially-orthogonal with a radial vector which extends out from a location on the pathology, even when the phone is moved (e.g. panned or spiraled) over and/or around the pathology. In an example, a method, device, and/or system for guiding a person concerning how to move a phone (or other imaging device) to record images of a pathology or injury (e.g. a wound or ulcer) on a portion (e.g. a foot) of a body can comprise a phone (or other imaging device), wherein the phone is configured to be moved along a virtual arcuate (e.g. spiral or helical) path in space to record images of a pathology (e.g. wound or ulcer) from different perspectives.

In an example, a person can be guided to move a phone (or other imaging device) in an oscillating, back-and-forth, sinusoidal, serpentine, spiral, helical, or starburst pattern along a virtual path or shape in order to record images of a pathology (or injury) on a body. In an example, guidance concerning how to move a phone (or other imaging device) to record a set of images of a pathology (or injury) can include instructions to move the phone in a spiral and/or helical path (or shape) in (three-dimensional) space to record images of the pathology (or injury) from different angles.

In an example, a method can guide a person concerning how to move a phone (or other imaging device) along a path (or shape) in 3D space in order to record in-focus images of a pathology (or injury) on a body from different angles, wherein this guidance comprises a series of tones, sounds, and/or beeps, and wherein the pitch or volume of the tones, sounds, and/or beeps varies (e.g. increases or decreases) with the distance of the phone from the path, thereby helping the person to move and keep the phone along the path.

In an example, a method can provide real-time visual guidance to a person moving a phone (or other imaging device), wherein this guidance helps the person to move the phone along a specified arcuate path (or shape) in space relative to a pathology (or injury) on a body, wherein recording images of the pathology from multiple locations along the path enables construction a digital 3D model of the pathology for medical evaluation, wherein a device emits light with a first color (or pulse frequency) when the phone is on the path, and wherein a device emits light with a second color (or pulse frequency) when the phone deviates from the path.

In another example, a method for guiding a person concerning how to move a phone (or other imaging device) to record images of a pathology (or injury) on a body can use sound, wherein the frequency, pitch, volume, or pulse rate of sound emitted from the phone changes if the phone deviates from a specified path (or shape) in 3D space over, across, and/or around the pathology. In an example, a method for helping a person to record high-quality images of a pathology (or injury) on a body for medical evaluation using a phone (or other imaging device) can include providing the person with auditory guidance concerning how to move the phone along a specified arcuate path (or shape) in 3D space over, across, and/or around the pathology; wherein this auditory guidance comprises generation of sounds whose pitch, tone, frequency, and/or pulse rate increases when the phone deviates from the path.

In another example, a method for helping a person to record high-quality images of a pathology (or injury) on a body for medical evaluation using a phone (or other imaging device) can include providing the person with visual guidance concerning how to move the phone along a specified arcuate path (or shape) in 3D space over, across, and/or around the pathology; wherein this visual guidance comprises written or graphical instructions which are triggered when the phone deviates from the path. In an example, auditory guidance can comprise sounds which guide a person concerning deviation of a phone (or other imaging device) from a desired movement path (or shape) in 3D space, wherein one or more of the following characteristics of the sound guide the person: constant sound volume, change in sound volume, constant sound pitch or frequency, change in sound pitch or frequency, constant sound pulse rate, change in sound pulse rate, constant sound waveform shape, and change in sound waveform shape.

In an example, in tactile or haptic guidance system, one or more characteristics (e.g. magnitude, duration, or pulse rate) of vibrations can be based on based on the direction and/or the amount of deviation of the location of a phone (or other imaging device) from a desired virtual path or shape in 3D space. In an example, sound emitted by a phone (or other imaging device) can have a lower pitch when the phone is on the correct path to record images of a pathology and can have a higher pitch when the phone deviates from the correct path. In an example, sound emitted by a phone (or other imaging device) can be quieter (or even silent) when the phone is on the correct path to record images of a pathology and can be louder when the phone deviates from the correct path.

In an example, a method can comprise using machine learning and/or artificial intelligence to generate auditory directional indicators (e.g. spoken directional instructions or tone sequences) which guide a person concerning how to move a phone (or other imaging device) along a specified path (or shape) in 3D space over, across, and/or around a pathology (or injury) on a body in order to record images of the pathology. In an example, a method can provide real-time auditory guidance to a person moving a phone (or other imaging device) to record image of a pathology (or injury) on a body, wherein a first set of tones (e.g. with a first frequency, pulse rate, or frequency progression) indicates that the phone is too close to the pathology, and wherein a second set of tones (e.g. with a second frequency, pulse rate, or frequency progression) indicates that the phone is too far from the pathology.

In another example, a method for guiding a person concerning how to move a phone (or other imaging device) to record images of a pathology (or injury) on a body on a body can comprise verbal cues (e.g. spoken words) from the phone, wherein the verbal cues (e.g. spoken words) tell the person which direction to move the phone, and wherein the verbal cues are generated by real-time analysis of images from the phone by machine learning and/or artificial intelligence. In an example, a method for guiding a person concerning how to move a phone (or other imaging device) to record images of a pathology (or injury) on a body on a body can comprise verbal cues (e.g. spoken words) from the phone.

In another example, a method for guiding a person concerning how to move a phone (or other imaging device) to record images of a pathology (or injury) on a body can include emission of sounds which guide the person concerning how to move the phone relative to the pathology in order to capture images of the pathology from different distances and angles. In an example, a method, device, or system can provide a person with real-time AI-generated auditory guidance concerning how to move a phone (or other imaging device) along a specified arcuate path (or shape) in 3D space over, across, and/or around a pathology (or injury) on a body. In an example, a system for recording images of a pathology (or injury) for medical evaluation can comprise placing a phone (or other imaging device) in an extendable arm of a mobile robot, wherein the robot emits one or more of the following sounds: beep-bleep-bop-beep-beep, beeple-beep-boop-beep, wooooah, bleep-bleep-beepy-beep-boop, and beep-bleeple-boop-boop-beep, and wherein the robot projects a holographic image of the pathology.

In an example, auditory guidance to guide a person concerning how to move a phone (or other imaging device) relative to a pathology (or injury) on a body can comprise providing the person with a series of spoken instructions in real time, wherein the spoken instructions are a function of actual movement of the phone in space compared to a desired movement path in space for the phone, and wherein the spoken instructions guide the person concerning how to move the phone onto the desired movement path and/or keep the phone on the desired movement path.

In an example, a method can comprise providing auditory (e.g. spoken words or sound tones), visual (e.g. written words or graphically-displayed objects), and/or tactile (e.g. vibrations) guidance to a person concerning how to move a phone (or other imaging device) in an arc over, across, and/or around a pathology (or injury) on a body. In an example, a method can comprise providing visual directional indicators (e.g. displayed arrows, pointers, or cursors) to a person concerning how to move a phone (or other imaging device) relative to a pathology (or injury) on a body to record useful medical images. In another example, a method can guide a person concerning how to move (or otherwise operate) a phone (or other imaging device) to record images of a pathology (or injury) on a body via visual guidance comprising written words, arrows or other directional indictors, graphic symbols, animations, light emission, laser beams, and/or displayed images.

In an example, a method for guiding a person concerning how to move a phone (or other imaging device) to record images of a pathology (or injury) on a body can include real-time visual guidance on the screen of the phone, wherein this visual guidance comprises directional arrows, vectors, or other graphic directional cues. In another example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has conic section shape.

In an example, a method for helping a person to record high-quality images of a pathology (or injury) on a body for medical evaluation using a phone (or other imaging device) can include providing the person with visual guidance concerning how to move the phone along a specified arcuate path (or shape) in 3D space over, across, and/or around the pathology. In an example, a visual display to guide a person concerning how to move a phone (or other imaging device) can comprise a bubble displayed on a computer screen, analogous to the air bubble in fluid in a physical level tool. In an example, guidance for a person concerning how to move a phone (or other imaging device) can include a cross-hairs display which indicates a desired position of the phone relative to pathology (or injury) on a body.

In an example, a method for guiding a person concerning how to move a phone (or other imaging device) to record images of a pathology (or injury) on a body can comprise instructing the person to move the phone so as to align two virtual objects (or patterns) which are shown in an augmented reality display. In an example, a system to guide the person concerning how to move or otherwise operate a phone (or other imaging device) to record images of a pathology (or injury) on a body can comprise: a phone which records images of the pathology; and an augmented reality display. In another example, visual guidance for how to move a phone (or other imaging device) can comprise displaying one or more virtual objects near a pathology (or injury) on a body in an augmented reality display, wherein the one or more virtual objects include one or more alignment guides (e.g. cross-hairs or target symbols) which are aligned when the phone is in the proper location and/or when the phone is on a desired path (or shape) in 3D space to record images of the pathology.

In an example, a method can guide a person concerning how to move (or otherwise operate) a phone (or other imaging device) to record images of a pathology (or injury) on a body via an augmented reality display which shows the pathology, the location of the phone (or other imaging device), and how the phone should be moved to record images of the pathology. In another example, a method can include providing a person with guidance concerning how to move a phone (or other imaging device) relative to a pathology (or injury) on a body in order to record useful images of the pathology for medical evaluation, wherein this guidance comprises aligning a virtual geometric pattern (e.g. a circle, cross hairs, or target symbol) over the pathology in an augmented reality display.

In an example, a method for guiding a person concerning how to move a phone (or other imaging device) to record images of a pathology (or injury) on a body can comprise instructing a person moving the phone to align a current view of the pathology with a virtual object (or pattern) in an augmented reality display. In an example, a method to guide a person concerning how to move a phone (or other imaging device) to record images of a pathology (or injury) on a body can comprise an augmented reality display which shows the pathology and a virtual object and/or pattern, wherein the person is guided to move the virtual object and/or pattern relative to the pathology in the augmented reality display. In an example, an augmented reality display can show: a pathology (or injury) on a body; a current location and past path in 3D space of a phone (or other imaging device); and an identified virtual path or shape in 3D space along which the phone should be moved to record images of the pathology, wherein the virtual path or shape is identified by machine learning and/or artificial intelligence.

In an example, an augmented reality display on a phone to guide a person concerning how to move the phone to record images of a pathology (or injury) can show one or more of the following views: a current view of the phone; a current view of the pathology; a view of a path (e.g. a virtual arcuate path or shape in 3D space) along which the phone should be moved to record images of the pathology from different perspectives; an oblique side view of the phone, the pathology, and the virtual path; an overhead (e.g. top down) view of the phone, the pathology, and the virtual path; a view of the pathology from the perspective of the phone (e.g. from the camera on the phone); one or more directional indicators which show which direction the phone should be moved now (e.g. relative to the path and/or the pathology); one or more directional indicators which show how the phone should be rotated or pivoted now (e.g. relative to the path and/or the pathology); an animation which shows the phone moving along the virtual path; and a view of which portions of the path have been traveled by the phone and which portions of the path have not yet been traveled by the phone.

In an example, guidance concerning how to move a phone (or other imaging device) to record a set of images of a pathology (or injury) can comprise a suggested phone movement path (or shape) in three-dimensional space which is superimposed on an image of the pathology (or injury) in an augmented reality display shown on the screen of the phone. In an example, visual guidance for how to move a phone (or other imaging device) can comprise the display of one or more virtual objects relative to a pathology (or injury) on a body in an augmented reality display, wherein this display is on the screen of the phone.

In another example, a method for guiding a person concerning how to move a phone (or other imaging device) to record images of a pathology (or injury) on a body can include using vibration of the phone to guide the person concerning how to move the phone along a specified path (or shape) in 3D space relative to the pathology, wherein movement of the phone along this path enables recording images from different angles and/or distances to create a 3D virtual model of the pathology. In an example, a method for guiding a person concerning how to move a phone (or other imaging device) to record images of a pathology (or injury) on a body can include using vibration of the phone to guide the person concerning how to move the phone relative to the pathology, wherein different vibration patterns and/or intensities guide the person to move the phone in different directions.

In another example, a method to guide a person concerning how to move a phone (or other imaging device) to record images of a pathology (or injury) on a body can comprise generation of vibration with a first frequency or magnitude when the phone is too far from the pathology, generation of vibration with a second frequency or magnitude when the phone is the proper distance from the pathology, and generation of vibration with a third frequency or magnitude when the phone is too close to the pathology. In an example, a method, device, or system can provide a person with real-time AI-generated tactile guidance concerning how to move a phone (or other imaging device) along a specified path (or shape) in 3D space over, across, and/or around a pathology (or injury) on a body, wherein a first vibration pattern guides the person to pivot or rotate the phone in a first direction and a second vibration pattern guides the person to pivot or rotate the phone in a second direction.

In an example, a person can be guided concerning how to move (or otherwise operate) a phone (or other imaging device) to record images of a pathology (or injury) by tactile and/or haptic guidance comprising vibrations and/or vibration sequences. In an example, guidance concerning how to move the phone (or other imaging device) to record images of a pathology (e.g. wound, ulcer, or skin lesion) on a body can be tactile (e.g. a vibration or sequence of vibrations).

In an example, a method can include providing guidance to a person concerning how to move a phone (or other imaging device) relative to a pathology (or injury) on a body in order to record useful images of the pathology for medical evaluation, wherein this guidance is in the form of directional objects (e.g. arrows) which are displayed on the phone and/or a secondary device (e.g. smart eyewear, a smart watch, or a second phone). In an example, an augmented reality display showing how to move a first phone can be displayed on the screen of a second phone or via other mobile device. In an example, guidance concerning how to move a phone (or other imaging device) to record a set of images of a pathology (or injury) can comprise a suggested phone movement path (or shape) in three-dimensional space which is superimposed on an image of the pathology (or injury) in an augmented reality display shown by a secondary device such as smart eyewear.

In another example, a method can comprise providing an augmented reality display (e.g. via a phone or smart eyewear) which visually guides a person concerning how to move a phone (or other imaging device) so as to virtually align a current image of a pathology (or injury) on a body with a previous image of the pathology. In an example, a method can comprise: (a) using augmented reality (e.g. shown on the screen of a phone, another mobile device, or smart eyewear) to display a virtual object (e.g. virtual cross-hairs, arrow, cursor, target symbol, or prior body image) which is superimposed on a location on a pathology (or injury) on a body; and (b) guiding the person to move a phone (or other imaging device) so that the virtual object is (and remains) aligned with that location on the pathology in the augmented reality display as the phone is moved over, across, and/or around the pathology.

In another example, a method for guiding a person concerning how to move a phone (or other imaging device) to record images of a pathology (or injury) on a body on a body can include a display (e.g. a screen on the phone or display via augmented reality eyewear) which shows: the pathology; the phone; and a virtual path or shape in 3D space over, across, and/or around the pathology along which the phone should be moved. In an example, visual guidance can be provided to a person by augmented reality via a phone screen or an eyewear display.

In an example, a method to guide a person concerning how to move a phone (or other imaging device) to record images of a pathology (or injury) on a body can comprise displaying a virtual path or shape in three-dimensional space via augmented reality eyewear, wherein the person is instructed to move the phone (or other imaging device) along the virtual path or shape. In an example, an augmented reality display showing how to move a phone can be displayed via eyewear. In an example, visual guidance for moving a phone can be shown on an (augmented reality) eyewear display.

In an example, a method for guiding a person concerning how to move a smart watch (or watch band) with a camera to record images of a pathology (or injury) can include computer-generated voice commands such as moving the watch closer, farther, right, left, forward, backward, clockwise, and counter-clockwise. In an example, visual guidance for how to move a phone (or other imaging device) can comprise the display of one or more virtual objects relative to a pathology (or injury) on a body in an augmented reality display, wherein this display is on the screen of a smart watch or wrist band. In another example, a method for guiding a person concerning how to move a phone (or other imaging device) to record images of a pathology (or injury) on a body on a body can include a display (e.g. on a phone, a smart watch, or augmented reality eyewear) which shows: the pathology; and a virtual path in 3D space over, across, and/or around the pathology along which the phone should be moved.

In an example, a method can comprise using machine-learning and/or artificial intelligence to combine a plurality of images of a pathology (or injury) on a body from difference distances and angles into a digital 3D model of the pathology for medical evaluation purposes, wherein a healthcare provider can view the digital 3D model from virtually from different distances and angles. In another example, an AI-guided second set of one or more images of a pathology (or injury) on a body can enable more accurate cand/or complete creation of a digital 3D model of a pathology than an unguided first set of one or more images. In an example, machine learning and/or artificial intelligence can be used to integrate multiple images of a pathology (or injury) on a body from different angles into a digital 3D model of the pathology.

In another example, a method can comprise using machine learning and/or artificial intelligence to analyze differences between an image of a pathology (or injury) at a first time and an image of the pathology at a second time (e.g. the present time) in order to evaluate changes in the size, area, depth, volume, shape, outline, texture, color, temperature, spectral absorption distribution, and/or movement of the pathology. In an example, a method for guiding a person concerning how to move a phone (or other imaging device) to record images of a pathology (or injury) on a body can comprise instructing a person moving the phone to align a current view of the pathology with a previous image of the pathology in an augmented reality display. In an example, machine learning and/or artificial intelligence can align images of a pathology (or injury) on a body which are recorded at different times (e.g. align the centers or outlines of the pathology recorded at different times) in order to create a (time series) video of changes in the pathology over time.

In an example, a method can comprise: instructing a person to find an object with a known (e.g. standardized) color and/or size in their environment and to record a first set of one or more images of the object using a phone (or other imaging device); receiving the first set of one or more images; using machine learning and/or artificial intelligence to analyze the first set of one or more images to determine whether the object can serve to calibrate the color and/or size of a pathology (or injury) on a body; if the object can serve for calibration of color and/or size, then instructing the person to record a second set of one or more images which includes both the object and the pathology; receiving the second set of one or more images; and using machine learning and/or artificial intelligence to analyze the second set of one or more images, including using the color and/or size of the object to calibrate the color and/or size of the pathology in the second set.

In an example, a method can use machine learning and/or artificial intelligence to adjust (e.g. calibrate) the size of a pathology (or injury) on a body in an image based on size of a nearby environmental object whose size is standardized and/or known. In another example, a method for guiding a person concerning how to use a phone (or other imaging device) to record images of a pathology (or injury) on a body can comprise instructing the person to enter the type and/or model of the phone into a user interface, wherein the type and/or model of the phone is used to calibrate the color spectrum of images recorded by the phone.

In an example, a set of images of a pathology (or injury) which is guided by artificial intelligence can be more accurate and/or more complete for medical evaluation than an unguided set of images for one or more of the following reasons: the AI-guided set shows the pathology in sharper focus; the AI-guided set shows the pathology with greater magnification; the AI-guided set shows the pathology from a wider range of angles; the AI-guided set shows the pathology from a greater range of distances; the AI-guided set includes a larger portion of the pathology; the AI-guided set has better illumination; and the AI-guided set includes an environmental object near the pathology for calibration of color and/or size.

In an example, a method for providing guidance concerning how to use a phone (or other imaging device) relative to a pathology (or injury) on a body can comprise: analyzing a first set of images of the pathology to evaluate ambient lighting; and guiding a person concerning how to change the level and/or type of ambient lighting if needed. In an example, a method to guide a person concerning how to use a phone (or other imaging device) to record images of a pathology (or injury) on a body can comprise: receiving a first set of one or more images of a pathology (or injury) on a body recorded by a phone (or other imaging device); analyzing the first set of one or more images using machine learning and/or artificial intelligence; and providing guidance to the person concerning how to adjust ambient conditions (e.g. lighting type or level) so as to record a second set of one or more images of the pathology, wherein the second set of one or more images enables more accurate diagnosis, characterization, and/or measurement of the pathology (or injury) than the first set of one or more images, and wherein the guidance is at least partly based on analysis of the first set of one or more images.

In another example, a method for guiding a person concerning how to use a phone (or other imaging device) to record images of a pathology (or injury) can comprise: receiving a first image of the pathology; analyzing the image to evaluate illumination level in the image; and guiding the person concerning how to adjust lighting (e.g. by moving to another location or changing/adding a nearby light source) if needed for a second image of the pathology. In an example, a method for guiding a person concerning how to use a phone (or other imaging device) to record images of a pathology (or injury) on a body can comprise instructing the person to change the light spectral filter of the phone.

In an example, a method can include enabling a (remote) data processor to (remotely) activate image recording when a phone (or other imaging device) is at a correct location and/or on a correct path (or shape) in 3D space from which to record images of a pathology (or injury) on a body. In an example, a method can include using machine learning and/or artificial intelligence to remotely control one or more functions of a phone (or other imaging device) to improve the quality of images of a pathology (or injury) on a body which are recorded by the phone.

In another example, a method for guiding a person concerning how to move a phone (or other imaging device) to record images of a pathology (or injury) on a body can comprise: receiving a live-stream of images from a phone (or other imaging device) at a remote data processor (e.g. a remote server or device); using machine learning and/or artificial intelligence to analyzing the images; and sending real-time (e.g. in less than one minute) feedback to the person concerning how they should move the phone to improve and/or supplement the images.

In an example, a method for guiding a person concerning how to move a phone (or other imaging device) to record images of a pathology (or injury) on a body can comprise: using a remote data processor (e.g. remote server or device) to receive a first sequence of phone images with a first level of usefulness (e.g. first level of focal accuracy); analyzing the images (e.g. using machine learning and/or artificial intelligence); and sending live (e.g. within one minute) feedback to the person concerning how they should move the phone to record a second sequence of phone images with a second level of usefulness (e.g. level of focal accuracy), wherein the second level is greater than the first level.

In an example, a method for using a phone (or other imaging device) to record images of a pathology (or injury) on a body can include remote control of one or more phone functions by artificial intelligence, wherein the one or more functions are selected from the group consisting of: change in focal distance; change in zoom; change in magnification level; change in color filter; change in illumination; change in exposure time; change in lens aperture; change in camera angle; and change in (video) image duration. In an example, if a phone (or other imaging device) has a functionality enabling remote adjustment of the focal direction of its camera (e.g. without moving the entire phone), then a method can include giving remote control of this functionality to a system (e.g. equipped with artificial intelligence) which enables remote adjustment of the focal angle and keeps the camera focus directed toward a pathology (or injury) even when the phone is moved.

In another example, a first person can have a pathology (or injury) on their body and a second person can receive guidance from a machine-learning or artificial intelligence system concerning how to move a phone (or other imaging device) over, across, and/or around the pathology to record images for medical evaluation. In an example, a person holding a phone (or other imaging device) which records images of a pathology (or injury) on a body can be the person whose body is imaged.

In another example, a pathology (or injury) can be a skin abnormality. In an example, a pathology can be a bed ulcer. In an example, a pathology can be a tissue abnormality. In an example, an injury can be a bruise. In an example, a method can comprise using machine learning and/or artificial intelligence to measure and/or evaluate the size, area, depth, volume, shape, outline, texture, color, temperature, spectral absorption distribution, and/or movement of a pathology (or injury) on a portion of a body. In an example, a machine learning and/or artificial intelligence model can be trained on anatomical images of pathologies, injuries, and/or tissue abnormalities on the bodies of a large number of different people. In an example, a method can use machine learning and/or artificial intelligence to analyze images of a pathology (or injury) on a body. In an example, a method can include using a data processor in a phone (or other imaging device) to analyze phone images of a pathology (or injury) on a body.

In another example, a method can comprise: (a) receiving a first set of one or more images of a pathology (or injury) on a body which have been recorded by a phone (or other imaging device); (b) analyzing the first set of images using machine learning and/or artificial intelligence to identify an arcuate path (or shape) in 3D space along which the phone should be moved to record a superior second set of one or more images of the pathology; and (c) generating visual directional indicators (e.g. graphical images or displayed words) which guide a person concerning move the phone along the identified path. In an example, a method can comprise: receiving a first set of images of a pathology (or injury) on a body which have been recorded by a phone (or other imaging device); using machine learning and/or artificial intelligence to identify problems with the first set of images which make them unsatisfactory for use in medical evaluation; and guiding a person concerning how to move the phone, change the operation of the phone, and/or change environmental conditions to correct the problems with the first set of images.

In another example, a method can use machine learning and/or artificial intelligence for: analyzing an unguided first set of images of a pathology (or injury) on a body; and guiding a person concerning how to move or otherwise operate a phone (or other imaging device) to record a guided second set of images of the pathology (or injury), wherein the second set is better than the first set for medical evaluation purposes. In an example, a method for imaging a pathology (or injury) on a body can comprise: receiving a first set of one or more images of a pathology (or injury) on a body recorded by a phone (or other imaging device); analyzing (e.g. using machine learning and/or artificial intelligence) the first set of one or more images to determine a path (or shape) in 3D space along which a phone should be moved to record a second set of one or more images of the pathology, wherein the second set corrects identified problems with the first set; and guiding a person concerning how to move the phone along the path (or shape) in 3D space to record the second set of one or more images.

In an example, a method to guide a person concerning how to move a phone (or other imaging device) to record images of a pathology (or injury) on a body can comprise: (a) receiving a first set of one or more images of a pathology (or injury) on a body recorded by a phone (or other imaging device); (b) analyzing the first set of one or more images in close to real-time (e.g. less than a couple seconds or minutes) using machine learning and/or artificial intelligence; and (c) using the results of analysis of the first set of one or more images to guide the person concerning how to move and/or operate the phone so as to record a second set of one or more images of the pathology, wherein the second set of one or more images enables more accurate diagnosis, characterization, and/or measurement of the pathology (or injury).

In an example, a method to guide a person concerning how to move a phone (or other imaging device) to record images of a pathology (or injury) on a body can comprise: asking a person to use a phone to record a first set of one or more images of the pathology; receiving the first set of one or more images from the phone; analyzing the first set of one or more images using machine learning and/or artificial intelligence; and providing guidance to the person concerning how to move the phone to record a second set of one or more images of the pathology, wherein the second set is better focused, more accurate, more comprehensive, and/or more useful for medical evaluation than the first set. In an example, a method to guide a person concerning how to operate a phone (or other imaging device) to record images of a pathology (or injury) on a body can comprise: receiving a first set of one or more images of a pathology on a body recorded by the phone (or other imaging device); analyzing the first set of one or more images; and guiding the person concerning how to operate the phone to record a second set of one or more images of the pathology.

In an example, a system for helping a person to record high-quality images of a pathology (or injury) on a body for medical evaluation using a phone (or other imaging device) can comprise: a phone; a camera on the phone; a data processor; and a visual display (e.g. screen and/or augmented reality display); wherein the data processor receives a first set of images of a pathology which has been recorded by the camera; wherein the data processor (e.g. using machine learning and/or artificial intelligence) analyzes the first set of images to determine an arcuate path (or shape) in 3D space along which the phone should be moved to record a superior second set of images of the pathology; and wherein the system uses the visual display to provide a person with real-time visual guidance (e.g. graphical objects or displayed words) concerning how to move the phone along the arcuate path in 3D space.

In another example, an AI-guided second set of one or more images of a pathology (or injury) on a body can enable more-accurate viewing of the pathology than an unguided first set of one or more images. In an example, machine learning and/or artificial intelligence analysis of a first set of phone (or other imaging device) images of a pathology (or injury) on a body can: estimate the distance and angle between the surface of the pathology and the phone; and determine a suggested path (or shape) in 3D space along which the phone should be moved to record a second set of phone images of the pathology.

In another example, a method can comprise: receiving a first set of images of a pathology (or injury) on a body which have been recorded by a phone (or other imaging device); using machine learning and/or artificial intelligence to identify problems with the first set of images which make them unsatisfactory for use in medical evaluation; and guiding a person concerning one or more actions which they should perform to record a second set of images of the pathology which correct the problems with the first set of images; wherein the one or more actions are selected from the group consisting of: moving the phone in a different direction; activating a light emitter on the phone; changing environmental lighting sources in the current environment allocation or moving to a different environmental location with different lighting sources; changing the angle and/or orientation of the phone; changing the distance between the phone and the pathology; changing the focal distance of the phone; and changing the zoom or magnification setting of the phone.

In another example, a method, device, or system can use machine learning and/or artificial intelligence to guide a person concerning how to record a second (guided) set of images of a pathology (or injury) on a body which are superior to those recorded in a first (unguided) set of images because the second set shows the pathology with greater magnification. In an example, a method, device, or system can use machine learning and/or artificial intelligence to guide a person concerning how to record a second (guided) set of images of a pathology (or injury) on a body which are superior to those recorded in a first (unguided) set of images because the second set includes a larger portion of the pathology.

In an example, a method can provide guidance to a person concerning how to move a phone (or other imaging device), how to adjust the operation of the phone, and/or how to change environmental conditions in the event that a first set of images of a pathology (or injury) has problems and cannot be used for medical evaluation), wherein this guidance is selected to correct one or more problems with the first set of images, and wherein the type of correction is selected from the group consisting of: changing the distance of the phone from the pathology because the first set of images are out of focus and/or have insufficient detail; changing the location and/or the angle of the phone relative to the pathology because the there is insufficient area of the first set of images do not capture enough of the pathology in the frame of the first set of images; and changing ambient lighting and/or activating phone-based illumination because the first set of images is too dim or too bright.

In an example, a method can use machine learning and/or artificial intelligence to provide a person with guidance concerning how to move a phone (or other imaging device) to record images of a pathology (or injury) on a body. In an example, a method can comprise guiding a person concerning how to move a phone (or other imaging device) in directions relative to their head (e.g. toward one's head, away from one's head, to one's right, or to one's left).

In an example, a method can comprise providing a real-time dialog between a person moving a phone (or other imaging device) and an AI-controlled system, wherein this real-time dialog guides the person concerning how to move the phone in real-time relative to a pathology (or injury) on a body in order to capture in-focus images of the pathology from different locations and/or angles, and wherein these images from different locations and/or angles are combined to create a digital 3D model of the pathology for later review and evaluation. In an example, a method for guiding a person concerning how to use a phone (or other imaging device) to record images of a pathology (or injury) on a body can comprise instructing the person to change the magnification setting of the phone.

In an example, a method for using a phone (or other imaging device) to record images of a pathology (or injury) on a body can include allowing a machine learning and/or artificial intelligence system to automatically adjust the focusing, zoom, magnification, and/or color filter functions of the phone. In an example, guidance to a person holding a phone (or other imaging device) which is recording images of a pathology (or injury) on a body can be provided in real time, wherein this guidance causes the phone to be moved in a path (or shape) in space which records in-focus images of the pathology from different angles.

In an example, a method can include providing a person with guidance concerning how to move a phone (or other imaging device) to record images of a pathology (or injury) on a body for medical evaluation, wherein this guidance comprises sounds or sound sequences, and wherein changes in the pitch, frequency, or pulse rate of the sounds or sound sequences tell the person how to move the phone (e.g. closer to the body or farther from the body). In another example, a method for guiding a person concerning how to move a phone (or other imaging device) to record images of a pathology (or injury) on a body can including the emission of sounds and/or tones, wherein different sounds and/or tones (e.g. high or low) or sound and/or tone sequences (e.g. ascending or descending) indicate that the phone should be moved in different directions (e.g. farther from the body or closer to the body).

In an example, a method to guide a person concerning how to move a phone (or other imaging device) to record images of a pathology (or injury) on a body can comprise a device which emits a first tone (e.g. with a first frequency or pitch) when the phone is too far from the pathology, a second tone (e.g. with a second frequency or pitch) when the phone is the proper distance from the pathology, or a third tone (e.g. with a third frequency or pitch) when the phone is too close to the pathology. In another example, a method with AI-based analysis of phone (or other imaging device) images of a pathology (or injury) on a body can include analysis of whether the images are in focus to determine whether the phone should be moved closer to (or farther away from) the pathology and provide appropriate guidance to a person holding and/or moving the phone.

In an example, a screen display or augmented reality display can show the distance between a phone (or other imaging device) and (the center of) a pathology (or injury) on a body. In an example, guidance concerning how to move a phone (or other imaging device) to record a set of images can include instructions to move the phone closer to a pathology (or injury) and/or move the phone farther from the pathology. In an example, sound emitted by a phone (or other imaging device) can have a higher pitch when the phone is too far from a pathology for accurate (e.g. in focus) imaging and can have a lower pitch when the phone is the correct distance from the pathology for accurate (e.g. in focus) imaging. In an example, sound emitted by a phone (or other imaging device) can have a lower pitch when the phone is too close to a pathology for accurate (e.g. in focus) imaging and can have a higher pitch when the phone is the correct distance from the pathology for accurate (e.g. in focus) imaging.

In an example, sound emitted by a phone (or other imaging device) can be louder when the phone is too far from a pathology for accurate (e.g. in focus) imaging and can be quieter (or even silent) when the phone is the correct distance from the pathology for accurate (e.g. in focus) imaging. In an example, sound emitted by a phone (or other imaging device) can be quieter (or even silent) when the phone is too close to a pathology for accurate (e.g. in focus) imaging and can be louder when the phone is the correct distance from the pathology for accurate (e.g. in focus) imaging.

In an example, a method can include providing a person with guidance concerning how to move a phone (or other imaging device) to record images of a pathology (or injury) on a body for medical evaluation, wherein this guidance comprises sounds or sound sequences, and wherein changes in the pitch, frequency, or pulse rate of the sounds or sound sequences tell the person how to rotate, pivot, or tilt the phone (e.g. change the roll, pitch, or yaw of the phone). In another example, a method for guiding a person concerning how to move a phone (or other imaging device) to record images of a pathology (or injury) on a body on a body can include a display showing two or more objects (e.g. virtual cross-hairs, virtual arrows, virtual circles, or images of the pathology at different times), wherein the objects move relative to each other when the phone is moved, and wherein the objects become aligned in the display when the phone is in the proper location and/or orientation for capturing images of the pathology.

In an example, a method for guiding a person concerning how to move a phone (or other imaging device) to record images of a pathology (or injury) on a body can comprise emitting different tones or tone sequences which indicate that the phone should be moved in different directions or rotated to different orientations. In another example, a method for guiding a person concerning how to move a phone (or other imaging device) to record images of a pathology (or injury) on a body can comprise emitting different computer-generated words or phrases which indicate that the phone should be moved in different directions or rotated to different orientations. In an example, a method for providing guidance concerning how to move a phone (or other imaging device) relative to a pathology (or injury) on a body can comprise guiding a person concerning how to pan, rotate, pivot, and/or tilt the phone to keep the phone's camera focal vector directed toward the pathology while the phone is moved over, across, and/or around the pathology.

In an example, a method, device, or system can provide a person with real-time AI-generated visual guidance concerning how to move a phone (or other imaging device) along a specified path (or shape) in 3D space over, across, and/or around a pathology (or injury) on a body, wherein a first visual pattern guides the person to pivot or rotate the phone in a first direction and a second visual pattern guides the person to pivot or rotate the phone in a second direction. In an example, a screen display or augmented reality display can show the rotation a phone (or other imaging device) relative to a virtual path (or shape) across, over, or around a pathology (or injury) on a body. In an example, visual guidance can indicate the direction that a phone (or other imaging device) should be rotated and/or pivoted relative to its current location and/or orientation.

In an example, a method can comprise using AI (e.g. machine learning and/or artificial intelligence) to display roll, pitch, and yaw directional indicators (e.g. in augmented reality) to guide a person concerning how to move a phone (or other imaging device) along a specified arcuate path (or shape) in 3D space to record in-focus images of a pathology (or injury) on a body from different perspectives. In an example, a method can comprise using machine learning and/or artificial intelligence to: (a) identify a path (e.g. a trajectory or sequence of orientations) in 3D space along which a phone (or other imaging device) should be moved to record images of a pathology (or injury) on a body from different perspectives, wherein the path includes a changing location of the center (e.g. the centroid) of the phone and a changing orientation (e.g. roll, pitch, and yaw) of the phone; and (b) provide auditory, visual, or tactile guidance to a person concerning how to move the phone along this path, wherein this guidance includes changes in the location and orientation (e.g. roll, pitch, and/or yaw) of the phone. In another example, a screen display or augmented reality display can show the yaw, roll, or pitch of a phone (or other imaging device) relative to a virtual path (or shape) across, over, or around a pathology (or injury) on a body.

In an example, a method can provide a person with iterative, close-to-real-time, guidance for how a phone (or other imaging device) should be moved relative to a pathology (or injury) on a body in order to record useful images for medical evaluation. In another example, a method for guiding a person concerning how to move a phone (or other imaging device) to record images of a pathology (or injury) on a body can comprise guiding the person to move the phone along the surface of a virtual arcuate path or shape in 3D space (which is over, across, and/or around the pathology), wherein this guidance comprises a plurality of incremental, sequential instructions and movements from one location (e.g. location, area, or sector) of the path or shape to another, until substantially the entire path or shape has been spanned.

In an example, a person holding a phone (or other imaging device) can be guided concerning how to move the phone relative to a pathology (or injury) on a body in a near-real-time iterative manner comprising multiple feedback cycles, wherein each feedback cycle comprises a data processor receiving an image of the pathology, analyzing the images, and recommending an action (e.g. moving the phone in a selected direction) for the person holding the phone, wherein each cycle takes less than minute. In another example, visual guidance for moving a phone (or other imaging device) can comprise (straight or arcuate) arrows, wherein the direction of the arrows shows the direction in which a phone (or other imaging device) should be moved, and wherein other attributes of the arrows (e.g. thickness, color, or discontinuous line) can show the relative distance by which the phone should be moved and/or the speed with which the phone should be moved.

In an example, the location of phone (or other imaging device) in 3D space can be determined by artificial intelligence analysis of data from a motion sensor on the phone (or other imaging device). In an example, a method can provide iterative, close to real-time, guidance to a person concerning how to move a phone (or other imaging device) along a specified path (or shape) in 3D space over, across, and/or around a pathology (or injury) on a body; wherein each cycle of guidance comprises (a) instructing the person how to move the phone for a short distance in a selected direction and then (b) determining (e.g. via data from a motion sensor on the device and/or changes in recorded images) that the phone has been successfully moved in this manner, before starting the next cycle.

In an example, a method for guiding a person concerning how to move a phone (or other imaging device) to record images of a pathology (or injury) on a body can comprise instructing the person to move the phone along a virtual arcuate path (or shape) in 3D space which is shown via an augmented reality display (e.g. on a phone screen or in augmented reality eyewear). In an example, a method, device, and/or system for recording images of a pathology or injury (e.g. a wound, ulcer, or skin lesion) on a portion of a body can comprise guiding a person concerning how to move a phone (or other imaging device), wherein the person moves the phone along a virtual arcuate path (or shape) in space to record images of the pathology or injury from different perspectives.

In an example, a method, system, or device can guide a person concerning the (relative) speed (e.g. faster or slower) that they should move a phone (or other imaging device) along a selected path (or shape) in 3D space to record images of a pathology (or injury) on a body. In an example, guidance concerning how to move a phone (or other imaging device) to record a set of images of a pathology (or injury) can include instructions to move the phone in an arcuate path (or shape) in (three-dimensional) space to record images of the pathology (or injury) from different angles.

In another example, providing guidance concerning how to move a phone (or other imaging device) can include identification of a virtual pathway (or shape) in 3D space along which the phone should be moved to record images of a pathology (or injury) on a body from multiple distances and/or angles, wherein this pathway (or shape) is identified by machine learning and/or artificial intelligence based on analysis of a first set of one or more images of the pathology. In an example, the movement path of a phone (or other imaging device) in 3D space over, across, and/or around a pathology (or injury) on a body can be within a flat plane. In another example, a method can comprise using machine learning and/or artificial intelligence to provide (auditory, visual, or tactile) guidance to a person concerning how to move a phone (or other imaging device) in an are over, across, and/or around a pathology (or injury) on a body, wherein this arc is centered on a location on the pathology.

In an example, a method to guide a person concerning how to move a phone (or other imaging device) to record images of a pathology (or injury) on a body can comprise instructions to the person to move the phone in one or more of the following directions: lateral movement of the phone along a plane which is generally parallel or tangential to the surface of the pathology; radial movement of the phone along a vector which extends out radially from the pathology; rotational movement of the phone around an axis which is generally parallel or tangential to the surface of the pathology; rotational movement of the phone around an axis which is generally perpendicular to the surface of the pathology; movement along an arcuate path (e.g. section of a circle or ellipse) across, over, or around the pathology; and movement along the arcuate surface of a concave 3D shape (e.g. section of a sphere or ellipsoid) across, over, or around the pathology.

In another example, a method for guiding a person concerning how to move a phone (or other imaging device) over, across, and/or around a pathology (or injury) on a body can comprise: creating an augmented reality display which shows the pathology, the phone, and a virtual arcuate shape in 3D space (e.g. section of a sphere or ellipsoid) along which the phone should be moved to record images of the pathology from multiple angles to create a digital 3D model of the pathology; and guiding the person concerning how to move the phone along this virtual arcuate shape (or surface) in 3D space.

In an example, a method for guiding a person concerning how to move a phone (or other imaging device) over, across, and/or around pathology (or injury) on a body can comprise: receiving images of the pathology which have been recorded by the phone; using machine learning and/or artificial intelligence to specify an arcuate 3D shape (e.g. a section of a sphere or ellipsoid) along which the phone should be moved to record images of the pathology from different perspectives (e.g. to create a digital 3D model of the pathology); and showing the person which areas of the arcuate 3D shape have been spanned (e.g. from which images have been recorded) by the phone and which areas remain to be spanned (e.g. from which images have not yet been recorded) by the phone.

In an example, a method for guiding a person concerning how to move a phone (or other imaging device) over, across, and/or around a pathology (or injury) on a body can comprise creating an augmented reality image which is shown to the person, wherein the augmented reality image includes: the pathology; the phone; and a virtual arcuate surface in 3D space (e.g. a section of a sphere or ellipsoid) over, across, and/or around the pathology; wherein the person is guided (e.g. with auditory, visual, or tactile guidance) concerning how to move the phone along the virtual arcuate surface in order to record images of the pathology from different perspectives (e.g. to create a digital 3D model of the pathology).

In an example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has a shape which is a section (e.g. half) of a sphere or ellipsoid. In an example, guidance concerning how to move a phone (or other imaging device) to record a set of images of a pathology (or injury) can include instructions to move the phone in a hemispherical path (or shape) in three-dimensional space to record images of the pathology (or injury) from different angles.

In an example, a method for record images of a pathology (or injury) on a portion of a body can comprise using artificial intelligence and augmented reality to guide a person concerning how to move the phone (or other imaging device) along the surface of an virtual arcuate path (or shape) in space over, across, and/or around the pathology, wherein the plane of the phone is kept substantially tangential to the surface of the virtual arcuate path (or shape). In an example, a method, device, or system can provide guidance to a person comprising (auditory, visual, or tactile) instructions concerning how to move a phone (or other imaging device) so that the intersection angle between a plane of the phone and a radial vector extending out from (the center of) a pathology (or injury) on a body is between 80 and 100 degrees. In another example, guidance concerning how to move a phone (or other imaging device) to record a set of images can include instructions to move the phone in a plane which is parallel to the surface of the pathology (or injury) (if the surface is relatively flat) or which is tangential to the surface of the pathology (if the surface is arcuate).

In an example, a method can guide a person holding a phone (or other imaging device) concerning how to move the phone back and forth (e.g. in an oscillating manner) over, across, and/or around a pathology (or injury) on a body in order to record in-focus images of the pathology from different angles. In another example, a method for guiding a person concerning how to move a phone (or other imaging device) to record images of a pathology (or injury) on a body can comprise an augmented reality display which shows a virtual object (e.g. pointer, cursor, target symbol, cross hairs, or other geometric pattern), an arcuate (e.g. circular, semi-circular, spiral, or helical) virtual path (or shape) in 3D space relative to the pathology, and the pathology, wherein the person is instructed to move the phone so as to move the virtual object along the virtual path.

In an example, a method for guiding a person concerning how to move a phone (or other imaging device) to record images of a pathology (or injury) on a body can comprise guiding the person to move the phone in a back-and-forth, oscillating, serpentine, sinusoidal, or spiral manner in 3D space over, across, and/or around the pathology. In an example, a method for guiding a person concerning how to move a phone (or other imaging device) to record images of a pathology (or injury) on a body can comprise guiding the person to move the phone back and forth over the surface of a virtual concave shape (e.g. a section of a sphere or ellipsoid) until substantially all of surface has been spanned (e.g. covered).

In an example, a method for recording images of a pathology (or injury) on a body can comprise: (a) receiving a first set of one or more images of a pathology (or injury) on a portion of a body which have been recorded by a phone (or other imaging device); (b) using machine learning and/or artificial intelligence to analyze the first set of one or more images and to specify a virtual arcuate (e.g. spiral or helical) path in space from which a second set of one or more images of the pathology should be recorded; and (c) guiding a person concerning how to move the phone along the virtual arcuate (e.g. spiral or helical) path in space to record the second set of one or more images.

In an example, a method for recording phone (or other imaging device) images of a pathology (or injury) on a body for medical evaluation can comprise guiding a person concerning how to move a phone along one or more circular, semi-circular, spiral, or helical paths in space. In an example, a path along which a phone is moved can be a spiral or helical path along the surface of a section of a sphere (e.g. a hemisphere), wherein this section is centered on a pathology (or injury). In an example, guidance concerning how to move a phone (or other imaging device) to record a set of images of a pathology (or injury) can include instructions to move the phone in a sinusoidal path (or shape) in (three-dimensional) space to record images of the pathology (or injury) from different angles.

In another example, a method can guide a person concerning how to move a phone (or other imaging device) along a path (or shape) in 3D space in order to record in-focus images of a pathology (or injury) on a body from different angles, wherein this guidance comprises emission of light or display of graphical objects with colors (e.g. wavelengths) that vary with the distance of the phone from the path, thereby helping the person to move and keep the phone along the path. In an example, a method can guide a person concerning how to move a phone (or other imaging device) along a path (or shape) in 3D space in order to record in-focus images of a pathology (or injury) on a body from different angles, wherein this guidance comprises a series of tones, sounds, and/or beeps, and wherein the pulse rate of the tones, sounds, and/or beeps varies (e.g. decreases) with the distance of the phone from the path in a manner analogous to a Geiger counter, thereby helping the person to move and keep the phone along the path.

In another example, a method for guiding a person concerning how to move a phone (or other imaging device) to record images of a pathology (or injury) on a body on a body can include a display showing two or more objects (e.g. virtual cross-hairs, virtual arrows, virtual circles, or images of the pathology at different times), wherein the objects move relative to each other when the phone is moved, and wherein the objects align with each other in the display when the phone moves along a selected path in 3D space from which to capture images and diverge from each other in the display when the phone deviates from the selected path.

In an example, a method for helping a person to record high-quality images of a pathology (or injury) on a body for medical evaluation using a phone (or other imaging device) can include providing the person with auditory guidance concerning how to move the phone along a specified arcuate path (or shape) in 3D space over, across, and/or around the pathology; wherein this auditory guidance comprises generation of verbal directional instructions when the phone deviates from the path. In an example, a method for helping a person to record high-quality images of a pathology (or injury) on a body for medical evaluation using a phone (or other imaging device) can include providing the person with auditory guidance concerning how to move the phone along a specified arcuate path (or shape) in 3D space over, across, and/or around the pathology; wherein this auditory guidance comprises generation of sounds whose pitch, tone, frequency, and/or pulse rate decreases when the phone deviates from the path.

In an example, a method for helping a person to record high-quality images of a pathology (or injury) on a body for medical evaluation using a phone (or other imaging device) can include providing the person with visual guidance concerning how to move the phone along a specified arcuate path (or shape) in 3D space over, across, and/or around the pathology; wherein this visual guidance comprises changes in the color, size, direction, or shape of virtual objects in a display when the phone deviates from the path. In an example, in a visual guidance system, one or more characteristics (e.g. symbol, direction, color, size, or alignment) of graphic elements can be based on the direction and/or the amount of deviation of the location of a phone (or other imaging device) from a desired virtual path or shape in 3D space.

In an example, sound emitted by a phone (or other imaging device) can have a faster pulse rate when the phone is on the correct path to record images of a pathology and can have a slower pulse rate when the phone deviates from the correct path. In an example, sound emitted by a phone (or other imaging device) can have a slower pulse rate when the phone is on the correct path to record images of a pathology and can have a faster pulse rate when the phone deviates from the correct path.

In another example, a method can comprise generating auditory directional indicators (e.g. sounds, sound sequences, or spoken words) which guide a person concerning move a phone (or other imaging device) relative to a pathology (or injury) on a body in order to record useful medical images. In an example, a method can comprising providing guidance to a person concerning how to move a phone (or other imaging device) to record images of a pathology (or injury) on a body; wherein the guidance is auditory; and wherein the guidance further comprises emitting a first sound (sequence) to instruct the person change the roll the phone, emitting a second sound (sequence) to instruct the person to change the pitch of the phone, and/or emitting a third sound (sequence) to instruct the person to change the yaw of the phone.

In another example, a method can provide real-time auditory guidance to a person concerning how to move a phone (or other imaging device) to record image of a pathology (or injury) on a body, wherein this guidance helps the person to move the phone along a specified path (or shape) in 3D space, wherein recording images from multiple locations along the path enables creation a digital 3D model of the pathology for medical evaluation, wherein a first set of tones (e.g. with a first frequency, pulse rate, or frequency progression) indicates that the phone is on the path, and wherein a second set of tones (e.g. with a second frequency, pulse rate, or frequency progression) indicates that the phone has deviated from the path.

In an example, a method for guiding a person concerning how to move a phone (or other imaging device) to record images of a pathology (or injury) on a body can include computer-generated voice commands such as moving a phone (or other imaging device) closer, farther, right, left, forward, backward, clockwise, and counter-clockwise. In an example, a method for guiding a person concerning how to move a phone (or other imaging device) to record images of a pathology (or injury) on a body can include (computer-generated) spoken words (e.g. from the phone or from another device). In an example, a method for guiding a person concerning how to move a phone (or other imaging device) to record images of a pathology (or injury) on a body can use sound patterns and/or auditory cues to guide the person. In an example, a person can be guided concerning how to move (or otherwise operate) a phone (or other imaging device) to record images of a pathology (or injury) by auditory guidance comprising spoken words, tones or sounds, and/or tone or sound sequences.

In an example, auditory guidance can comprise sounds which guide a person concerning which direction to move a phone (or other imaging device), wherein one or more of the following characteristics of the sound guide the person concerning how to move the phone: constant sound volume, change in sound volume, constant sound pitch or frequency, change in sound pitch or frequency, constant sound pulse rate, change in sound pulse rate, constant sound waveform shape, and change in sound waveform shape. In another example, guidance concerning how to move a phone (or other imaging device) to record images of a pathology (e.g. wound, ulcer, or skin lesion) on a body can be auditory (e.g. sounds, sound sequences, or spoken words).

In an example, a method can comprise providing guidance to a person concerning how to move a phone (or other imaging device) to record images of a pathology (or injury) on a body; wherein the guidance is visual; and wherein the guidance further comprises displaying a first vector (e.g. arrow) to instruct the person change the roll the phone, displaying a second vector (e.g. arrow) to instruct the person to change the pitch of the phone, and/or displaying a third vector (e.g. arrow) to instruct the person to change the yaw of the phone. In another example, a method can comprise using machine learning and/or artificial intelligence to guide a person concerning how to move a phone (or other imaging device) whose screen displays a virtual object (e.g. a virtual cursor, target symbol, or cross hairs) superimposed on a pathology (or injury) on a body.

In an example, a method can provide real-time visual guidance to a person moving a phone (or other imaging device), wherein this guidance helps the person to move the phone along a specified arcuate path (or shape) in space relative to a pathology (or injury) on a body, wherein recording images of the pathology from multiple locations along the path enables construction a digital 3D model of the pathology for medical evaluation, wherein a device emits light or displays an object with a first color when the phone is too close to the pathology, and wherein a device emits light or displays an object with a second color when the phone is too far from the pathology.

In an example, a method for guiding a person concerning how to move a phone (or other imaging device) to record images of a pathology (or injury) on a body can include real-time visual guidance on the screen of the phone or a display in smart eyewear. In an example, a method for guiding a person concerning how to move a phone (or other imaging device) to record images of a pathology (or injury) on a body on a body can comprise identifying (and displaying) a virtual arcuate path (or shape) in 3D space along which the phone should be moved to record images of the pathology, wherein the path has a shape which is a section (e.g. arc) of a circle or ellipse.

In an example, a method, device, or system can provide a person with real-time AI-generated visual guidance concerning how to move a phone (or other imaging device) along a specified arcuate path (or shape) in 3D space over, across, and/or around a pathology (or injury) on a body. In an example, a visual display to guide a person concerning how to move a phone (or other imaging device) can comprise a bubble displayed on a computer screen, analogous to the air bubble in fluid in a physical level tool, wherein the bubble appears in the correct location on the computer screen when the phone is in the correct location (and/or on the correct path) in 3D space. In an example, visual guidance for moving a phone (or other imaging device) can comprise (straight or arcuate) arrows showing the direction in which a phone (or other imaging device) should be moved.

In another example, a method for guiding a person concerning how to move a phone (or other imaging device) relative to a pathology (or injury) on a body can use the juxtaposition of two virtual objects in an augmented reality display to guide the person concerning how to move the phone, wherein the person is instructed to move the phone until the two virtual objects are aligned in the augmented reality display. In an example, guidance concerning how a person should move a phone (or other imaging device) to record images of a pathology (or injury) can be provided via an augmented reality display. In another example, visual guidance for how to move a phone (or other imaging device) can comprise displaying one or more virtual objects near a pathology (or injury) on a body in an augmented reality display, wherein the one or more virtual objects include a virtual path (or shape) in 3D space along which the phone should be moved to record images of the pathology from different locations and/or angles.

In an example, a method can include providing an augmented reality display which shows a pathology (or injury) on a portion of a body, a first virtual object or pattern which remains stationery relative to the pathology as the phone (or other imaging device) is moved, and a second virtual object or pattern which moves relative to the pathology as the phone (or other imaging device) is moved. In an example, a method for guiding a person concerning how to move a phone (or other imaging device) to record images of a pathology (or injury) on a body can comprise an augmented reality display which shows a virtual object (e.g. pointer, cursor, target symbol, cross hairs, or other geometric pattern), a virtual path (or shape) in 3D space relative to the pathology, and the pathology, wherein the person is instructed to move the phone so as to move the virtual object along the virtual path.

In an example, a method for guiding a person concerning how to move a phone (or other imaging device) to record images of a pathology (or injury) on a body can comprise providing the person with an augmented reality display which shows a virtual pointer, cursor, target symbol, cross hairs, or another geometric pattern relative to the pathology. In an example, a screen display or augmented reality display can show the radial vector and/or position of a phone (or other imaging device) relative to (the center of) a pathology (or injury) on a body. In an example, an augmented reality display can show: a pathology (or injury) on a body; a current location and past path in 3D space of a phone (or other imaging device); and an identified virtual path or shape in 3D space along which the phone should be moved to record images of the pathology, wherein differences between (a) the current location and the past path of the phone and (b) the virtual path or shape are highlighted in the augmented reality display.

In an example, an augmented reality display to guide a person concerning how to move a phone (or other imaging device) to record images of a pathology (or injury) can show one or more of the following views: a current view of the phone; a current view of the pathology; a view of a path (e.g. a virtual arcuate path or shape in 3D space) along which the phone should be moved to record images of the pathology from different perspectives; an oblique side view of the phone, the pathology, and the virtual path; an overhead (e.g. top down) view of the phone, the pathology, and the virtual path; a view of the pathology from the perspective of the phone (e.g. from the camera on the phone); one or more directional indicators which show which direction the phone should be moved now (e.g. relative to the path and/or the pathology); one or more directional indicators which show how the phone should be rotated or pivoted now (e.g. relative to the path and/or the pathology); an animation which shows the phone moving along the virtual path; and a view of which portions of the path have been traveled by the phone and which portions of the path have not yet been traveled by the phone. In another example, guidance concerning how to move a phone (or other imaging device) to record a set of images can comprise a virtual geometric pattern (e.g. a target or cross-hairs pattern) which is shown relative to the pathology (or injury) on a body in an augmented reality display.

In an example, a method can comprise using machine learning and/or artificial intelligence to provide haptic and/or tactile sensations on a handheld or wearable device to a person holding a phone (or other imaging device) in order to guide the person concerning how to move the phone relative to a pathology (or injury) on a body which phone images, wherein a first pattern of haptic and/or tactile sensations indicates that the person should move the phone closer to the pathology and a second pattern of haptic and/or tactile sensors indicates that the person should move the phone farther from the pathology. In another example, a method for guiding a person concerning how to move a phone (or other imaging device) to record images of a pathology (or injury) on a body can comprise providing different vibrations or vibration sequences which indicate that the phone should be moved in different directions or rotated to different orientations.

In an example, a method to guide a person concerning how to move a phone (or other imaging device) to record images of a pathology (or injury) on a body can comprise providing vibrations or vibration sequences which guide a person concerning how to move a phone (or other imaging device), wherein (changes in) the frequency and/or amplitude of the vibrations guides the person concerning whether to pivot and/or rotate the phone. In an example, a method to guide a person concerning how to move a phone (or other imaging device) to record images of a pathology (or injury) on a body can comprise providing vibrations or vibration sequences which guide a person concerning how to move a phone (or other imaging device), wherein (changes in) the frequency and/or amplitude of the vibrations guides the person concerning whether to move the phone to the right or left.

In an example, a method, device, or system can provide a person with real-time AI-generated tactile guidance concerning how to move a phone (or other imaging device) along a specified path (or shape) in 3D space over, across, and/or around a pathology (or injury) on a body, wherein a first vibration pattern guides the person to move the phone closer to the pathology and a second vibration pattern guides the person to move the phone farther from the pathology. In an example, a person can be provided with tactile guidance (e.g. vibration patterns) concerning how to move a phone along a virtual arcuate path to record images of a pathology on a body.

In an example, guidance to a person concerning how to move a phone (or other imaging device) to capture images of a pathology (or injury) on a body can be selected from the group consisting of: augmented reality display, animation, color-changing lights, computer-generated speech, directional arrows, display of a virtual path for the phone to follow, displayed words, graphical interface, short video, sounds or tones, spoken instructions, text message, and vibrations.

In another example, a method for guiding a person to record images of a pathology (or injury) on a body can comprise: receiving images of a pathology (or injury) on a body recorded by a first phone (or other imaging device); and providing visual guidance to a person concerning how to move the first phone, wherein this guidance is provided via a screen on a second phone (or other imaging device). In an example, guidance concerning how to move a phone (or other imaging device) to record a set of images of a pathology (or injury) can comprise a suggested phone movement path (or shape) in three-dimensional space which is superimposed on an image of the pathology (or injury) in an augmented reality display shown on the screen of a secondary device such as a second phone or a smart watch. In another example, the location of phone (or other imaging device) in 3D space can be determined by artificial intelligence analysis of images of the phone (or other imaging device) recorded by a secondary imaging device (e.g. a second phone or camera-enabled eyewear).

In an example, a method can comprise: (a) using augmented reality (e.g. via a phone screen or an eyewear display) which shows a pathology (or injury) on a body; a phone (or other imaging device) near the pathology; a virtual path (or shape) in 3D space over, across, and/or around the pathology; and a virtual object on the virtual path; and (b) guiding a person to move the phone into alignment with the virtual object (as the object moves) to record images of the pathology from multiple perspectives along the virtual path (or shape). In an example, a method for guiding a person concerning how to move a phone (or other imaging device) to record images of a pathology (or injury) on a body can comprise providing a person with an augmented reality display on the phone or smart eyewear which shows a virtual pointer, cursor, target symbol, cross hairs, or another geometric pattern relative to the pathology.

In an example, a method, device, and/or system for recording images of a pathology or injury (e.g. a wound, ulcer, or skin lesion) on a portion of a body can comprise using augmented reality (e.g. an augmented reality display on a phone or smart eyewear) to guide a person concerning how to move a phone (or other imaging device), wherein the person moves the phone along a virtual arcuate path (or shape) in space to record images of the pathology or injury from different perspectives. In an example, a method for guiding a person concerning how to move a phone (or other imaging device) to record images of a pathology (or injury) on a body can comprise an augmented reality display on eyewear which shows the phone, the pathology, and a virtual path (or shape) in 3D space relative to the pathology, wherein the person is instructed to move the phone along the virtual path.

In an example, an augmented reality display in smart eyewear to guide a person concerning how to move a smart ring (with a camera) to record images of a pathology (or injury) can show one or more of the following views: a current view of the smart ring; a current view of the pathology; a view of a path (e.g. a virtual arcuate path or shape in 3D space) along which the smart ring should be moved to record images of the pathology from different perspectives; an oblique side view of the smart ring, the pathology, and the virtual path; an overhead (e.g. top down) view of the smart ring, the pathology, and the virtual path; a view of the pathology from the perspective of the smart ring (e.g. from the camera on the smart ring); one or more directional indicators which show which direction the smart ring should be moved now (e.g. relative to the path and/or the pathology); one or more directional indicators which show how the smart ring should be rotated or pivoted now (e.g. relative to the path and/or the pathology); an animation which shows the smart ring moving along the virtual path; and a view of which portions of the path have been traveled by the smart ring and which portions of the path have not yet been traveled by the smart ring.

In another example, visual guidance concerning moving a phone (or other imaging device) can be provided by augmented reality eyewear so that a person can continue to see the guidance having to contort their head to follow the phone display as the phone moves around a pathology (or injury). In an example, a method for guiding a person concerning how to move a smart watch (or watch band) with a camera to record images of a pathology (or injury) on a body can comprise providing the person with visual guidance (e.g. directional indicators or animations) which indicate whether the person should move the watch closer to the pathology or farther from the pathology.

In another example, a method for guiding a person concerning how to move a smart watch (or watch band) with a camera to record images of a pathology (or injury) on a body can comprise providing the person with tactile guidance (e.g. vibrations or vibration sequences) which indicate whether the person should move the watch closer to the pathology or farther from the pathology. In an example, visual guidance for moving a phone can be shown on a smart watch display. In an example, a method for guiding a person concerning how to move a phone (or other imaging device) to record images of a pathology (or injury) on a body on a body can include a display (e.g. on a phone, a smart watch, or augmented reality eyewear) which shows: an oblique view of the pathology; and how the phone should be moved relative to the pathology.

In an example, a method for guiding a person concerning how to move a phone (or other imaging device) to record images of a pathology (or injury) on a body can comprise guiding a person to record a plurality of images from different angles (and distances), wherein these images are integrated (e.g. by machine learning and/or artificial intelligence) to create a digital 3D model of the pathology which can be viewed by a healthcare provider from different angles (and distances). In an example, creation of a digital 3D model of a pathology (or injury) on a body can enable a variety of viewing perspectives (e.g. viewing angles and distances) by a healthcare provider, including simulated perspectives which extrapolate between viewing angles and distances in recorded images. In another example, machine learning and/or artificial intelligence can be used to integrate multiple images of a pathology (or injury) on a body from different angles and/or perspectives into a 3D virtual model of the pathology, wherein a healthcare provider can navigate and view this 3D virtual model from different angles and/or perspectives at a later time.

In an example, a method can comprise using machine-learning and/or artificial intelligence to align and combine images of a pathology (or injury) on a body which are recorded at different times into a time-lapse video of the pathology for medical evaluation purposes. In an example, a method for guiding a person concerning how to move a phone (or other imaging device) to record images of a pathology (or injury) on a body can comprise an augmented reality display, wherein this display superimposes an earlier view of the pathology over a current view of the pathology, and wherein the person is instructed to move the phone so as to align the two views in the augmented reality display.

In an example, a guided second set of images of a pathology (or injury) can be more accurate and/or more complete for medical evaluation than an unguided first set of images for one or more of the following reasons: the second set shows the pathology in sharper focus; the second set shows the pathology with greater magnification; the second set shows the pathology from a wider range of angles; the second set shows the pathology from a greater range of distances; the second set includes a larger portion of the pathology; the second set has better illumination; and the second set includes an environmental object near the pathology for calibration of color and/or size.

In an example, a method can including asking a person if they have an object available with a known color spectrum (e.g. a charge card, a standardized product label, a brand-name highlighter marker, a laser pointer with known wavelength, specific paint tone sample, or specific color test strip) which can help to calibrate the color of images of a pathology (or injury) recorded by a phone (or other imaging device). In an example, a method can use machine learning and/or artificial intelligence to adjust (e.g. calibrate) the color spectrum an image of a pathology (or injury) on a body based on color spectrum of a nearby environmental object whose color spectrum is standardized and/or known. In another example, a method for guiding a person concerning how to use a phone (or other imaging device) to record images of a pathology (or injury) can comprise instructing the person: to find an object in their environment with a known (e.g. standardized or consistent) color spectrum and/or a known (e.g. standardized or consistent) size; and to place the object near the pathology before recording an image of the pathology.

In an example, a method for guiding a person concerning how to move a phone (or other imaging device) to record images of a pathology (or injury) on a body can comprise instructing the person to activate light emission from the phone or another mobile device. In an example, a method for providing guidance concerning how to use a phone (or other imaging device) relative to a pathology (or injury) on a body can comprise: using machine learning and/or artificial intelligence to analyze a first set of images of the pathology to evaluate ambient lighting; and guiding a person concerning how to change the level and/or type of ambient lighting if needed. In an example, a method, device, or system can use machine learning and/or artificial intelligence to guide a person concerning how to record a second (guided) set of images of a pathology (or injury) on a body which are superior to those recorded in a first (unguided) set of images because the second set has better illumination. In an example, a method for guiding a person concerning how to use a phone (or other imaging device) to record images of a pathology (or injury) on a body can comprise instructing the person to move to a different environmental location with a different level and/or type of ambient light.

In another example, a method can comprise: receiving a first set of images of a pathology (or injury) on a body which have been recorded by a phone (or other imaging device); using machine learning and/or artificial intelligence to identify problems with the first set of images which make them unsatisfactory for use in medical evaluation; and using remote control of phone functions to perform one or more actions which correct the problems with the first set of images when a second set of images is recorded; wherein the one or more actions are selected from the group consisting of: activating a light emitter on the phone; changing environmental lighting sources in the current environment allocation or moving to a different environmental location with different lighting sources; changing the angle and/or orientation of the phone; changing the distance between the phone and the pathology; changing the focal distance of the phone; and changing the zoom or magnification setting of the phone.

In an example, a method can include using a data processor in a remote (but data-transmission-connected) device to analyze phone (or other imaging device) images of a pathology (or injury) on a body. In an example, a method can include: using machine learning and/or artificial intelligence to (automatically and/or remotely) activate a phone (or other imaging device) to start recording images of a pathology (or injury) on a body when the phone is at a specified location and/or moving along a specified path (or shape) in 3D space; and ending image recording when the phone deviates from the location and/or the path.

In an example, a method for guiding a person concerning how to move a phone (or other imaging device) to record images of a pathology (or injury) on a body can comprise: using a remote data processor (e.g. remote server or device) to receive a live-stream of images from a phone (or other imaging device); analyzing the images (e.g. using machine learning and/or artificial intelligence) in real time; and sending live feedback to the person (e.g. within one minute) concerning how they should next move the phone; wherein this method can be described as a real-time iterative process of sending images from a phone to a remote device and receiving guidance concerning how to next move the phone.

In an example, a method for recording images of a pathology (or injury) on a body can comprise: (a) receiving a first set of one or more images of a pathology (or injury) on a body which have been recorded by a phone (or other imaging device); (b) using machine learning and/or artificial intelligence to analyze the first set of one or more images and to specify a virtual arcuate path (or shape) in space from which a second set of one or more images of the pathology should be recorded; and (c) remotely controlling a (personal care or home) robot to move the phone along the virtual arcuate path in space to record the second set of one or more images.

In another example, a method, device, and/or system for recording images of a pathology or injury (e.g. a wound, ulcer, or skin lesion) on a portion of a body can comprise remotely controlling a (home and/or personal care) robot to move a phone (or other imaging device), wherein the robot moves the phone along a virtual arcuate path (or shape) in space to record images of the pathology or injury from different perspectives. In an example, images recorded by a phone (or other imaging device) can be (wirelessly) transmitted to a remote data processor (e.g. in a remote server or other device), wherein analysis of the images occurs in the remote data processor. In an example, a person can move a phone (or other imaging device) to record images of a pathology (or injury) on someone else's body for medical evaluation. In an example, a person who moves a phone to record images of a pathology (or injury) on a body can be different than the person who has the pathology on a body.

FIG. 1 shows an example of a method, device, and/or system for recording images of a pathology or injury (e.g. a wound, ulcer, or skin lesion) on a portion (e.g. foot) of a body comprising: a phone (or other imaging device) 101; wherein the phone is configured to be moved along a virtual arcuate path (or shape) 102 in space to record images of the pathology or injury (e.g. wound, ulcer, or skin lesion) 103 on the portion (e.g. foot) 104 of the body from different perspectives. In this figure, the virtual arcuate path (or shape) is shown as a wireframe surface.

The upper portion of FIG. 1 shows this method, device, or system at a first time when the phone (or other imaging device) is at a first location on the virtual arcuate path (or shape). The lower portion of FIG. 1 shows this method, device, or system at a second time when the phone is at a second location on the virtual arcuate path (or shape). Movement of the phone from the first location to the second location is shown by a dashed-line arrow which is over part of the virtual arcuate path (or shape). In an example, the phone can be moved (e.g. back and forth in an undulating, serpentine, and/or spiral pattern) to span the entire surface of the virtual arcuate path (or shape). In an example, images of the pathology (or injury) from multiple perspectives can be integrated (e.g. by machine learning and/or artificial intelligence) into a digital 3D model of the pathology for diagnostic and/or therapeutic purposes.

In an example, a method corresponding to FIG. 1 for recording images of a pathology (or injury) on a body can comprise: (a) receiving a first set of one or more images of a pathology (or injury) on a body which have been recorded by a phone (or other imaging device); (b) using machine learning and/or artificial intelligence to analyze the first set of one or more images and to specify a virtual arcuate path (or shape) in space from which a second set of one or more images of the pathology should be recorded; and (c) guiding a person concerning how to move the phone along the virtual arcuate path (or shape) in space to record the second set of one or more images.

In an example, the second set of one or more images can be superior for medical evaluation purposes than the first set of one or more images. In an example, the second set of one or more images can correct problems with the first set of one or more images such as poor focus, limited field of view, limited range of angles, and/or poor illumination. In an example, the second set of images can be used to create a digital 3D model of the pathology (or injury) on a body. In an example, guidance concerning how to move the phone (or other imaging device) can be auditory (e.g. sounds, sound sequences, or spoken words). In an example, guidance concerning how to move the phone (or other imaging device) can be visual (e.g. directional indicators or words shown on a screen and/or in augmented reality). In an example, guidance concerning how to move the phone (or other imaging device) can be tactile (e.g. a vibration or sequence of vibrations). Relevant variations discussed elsewhere in this disclosure or in priority-linked disclosures can also be applied to this example.

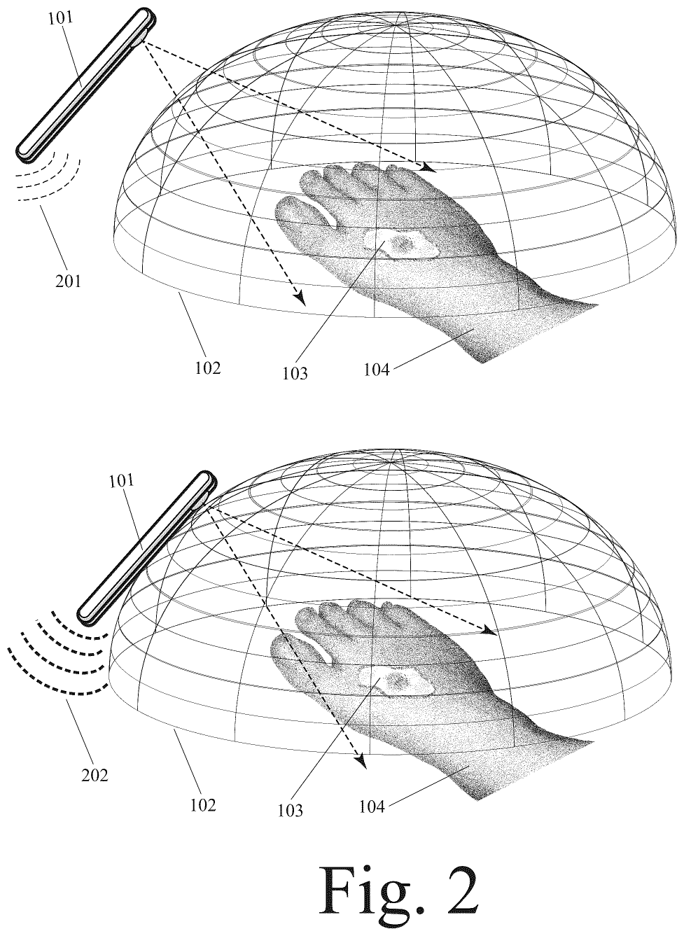

FIG. 2 shows an example of a method of a method, device, and/or system for guiding a person concerning how to move a phone (or other imaging device) to record images of a pathology or injury (e.g. a wound, ulcer, or skin lesion) on a portion (e.g. foot) of a body comprising: a phone (or other imaging device) 101; wherein the phone is configured to be moved along a virtual arcuate path (or shape) 102 in space to record images of the pathology or injury (e.g. wound, ulcer, or skin lesion) 103 on the portion (e.g. foot) 104 of the body from different perspectives; a first type or level of sound 201 which indicates that the phone is not on the path (or shape); and a second type or level of sound 202 which indicates that the phone is on the path (or shape). In this figure, the virtual arcuate path (or shape) is shown as a wireframe surface.

In this example, emitted sound is softer when the phone is off the correct path and louder when the phone is on the correct path. In this example, the first and second sounds provide auditory guidance to help the person keep the phone along the correct path to record images of the pathology. In an example, the location of the phone relative to the path can be determined (e.g. by machine learning and/or artificial intelligence) based on analysis of images from the phone and/or data from a motion sensor on the phone.

The upper portion of FIG. 2 shows this method, device, or system at a first time when the phone (or other imaging device) is not on the virtual arcuate path (or shape) and the first type or level of sound is emitted. The lower portion of FIG. 2 shows this method, device, or system at a second time when the phone is on the virtual arcuate path (or shape) and the second type or level of sound is emitted.

In an example, a method corresponding to FIG. 2 for recording images of a pathology (or injury) on a body can comprise: (a) receiving a first set of one or more images of a pathology (or injury) on a body which have been recorded by a phone (or other imaging device); (b) using machine learning and/or artificial intelligence to analyze the first set of one or more images and to specify a virtual arcuate path (or shape) in space from which a second set of one or more images of the pathology (or injury) should be recorded; and (c) providing the person with auditory guidance concerning how to keep along the virtual arcuate path as the phone (or other imaging device) is moved to record the second set of one or more images.

In this example, a person receives auditory guidance (e.g. emitted sounds or spoken words) concerning how to move a phone along a virtual arcuate path to record images of a pathology on a body. In another example, a person can receive visual guidance (e.g. displayed directional indicators or written words) concerning how to move a phone along a virtual arcuate path to record images of a pathology on a body. In an example, visual guidance can be provided via augmented reality on a phone screen or by an eyewear display. In another example, a person can receive tactile guidance (e.g. vibration patterns) concerning how to move a phone along a virtual arcuate path to record images of a pathology on a body. Relevant variations discussed elsewhere in this disclosure or in priority-linked disclosures can also be applied to this example.

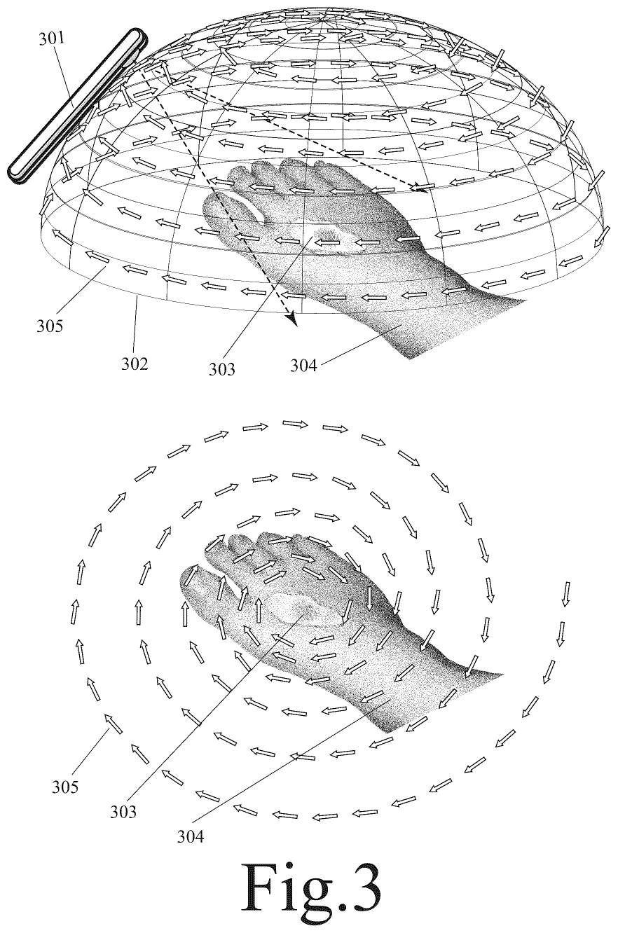

FIG. 3 shows an example of a method, device, and/or system for guiding a person concerning how to move a phone (or other imaging device) to record images of a pathology or injury (e.g. a wound, ulcer, or skin lesion) on a portion (e.g. a foot) of a body comprising: a phone (or other imaging device) 301; wherein the phone is configured to be moved along a virtual arcuate (e.g. spiral or helical) path 305 on the surface of a virtual section 302 of a sphere (e.g. hemisphere) to record images of a pathology or injury 303 (e.g. wound, ulcer, or skin lesion) on the portion (e.g. foot) 304 of a body from different perspectives. In this example, the path along which the phone is moved is a spiral or helical path along the surface of a section of a sphere (e.g. a hemisphere). In this example, this section is centered on a location on the pathology or injury. In this figure, the spiral or helical path along which the phone travels is represented by a series of arrows.

The upper portion of FIG. 3 shows an oblique side view of this method, device, and/or system, including an oblique side view of the virtual spiral or helix on the surface of a virtual hemisphere. The upper portion shows the phone (or other imaging device), the pathology or injury (e.g. wound, ulcer, or skin lesion), the portion of the body (e.g. foot), the section (e.g. hemisphere) of a sphere, and the virtual arcuate (e.g. spiral or helical) path on the virtual hemisphere along which the phone is moved. The lower portion of FIG. 3 shows a top-down view of this method, device, and/or system. The lower portion does not include the phone or the hemisphere so that the spiral or helical path can be seen more clearly.

In an example, a method corresponding to FIG. 3 for recording images of a pathology (or injury) on a body can comprise: (a) receiving a first set of one or more images of a pathology (or injury) on a portion of a body which have been recorded by a phone (or other imaging device); (b) using machine learning and/or artificial intelligence to analyze the first set of one or more images and to specify a virtual arcuate (e.g. spiral or helical) path in space from which a second set of one or more images of the pathology should be recorded; and (c) guiding a person concerning how to move the phone along the virtual arcuate (e.g. spiral or helical) path in space to record the second set of one or more images.

In an example, the second set of one or more images can be superior for medical evaluation purposes than the first set of one or more images. In an example, the second set of one or more images can correct problems with the first set of one or more images such as poor focus, limited field of view, limited range of angles, and/or poor illumination. In an example, the second set of images can be used to create a digital 3D model of the pathology (or injury) on a body. In an example, guidance concerning how to move the phone (or other imaging device) can be auditory (e.g. sounds, sound sequences, or spoken words). In an example, guidance concerning how to move the phone (or other imaging device) can be visual (e.g. directional indicators or words shown on a screen and/or in augmented reality). In an example, guidance concerning how to move the phone (or other imaging device) can be tactile (e.g. a vibration or sequence of vibrations). Relevant variations discussed elsewhere in this disclosure or in priority-linked disclosures can also be applied to this example.

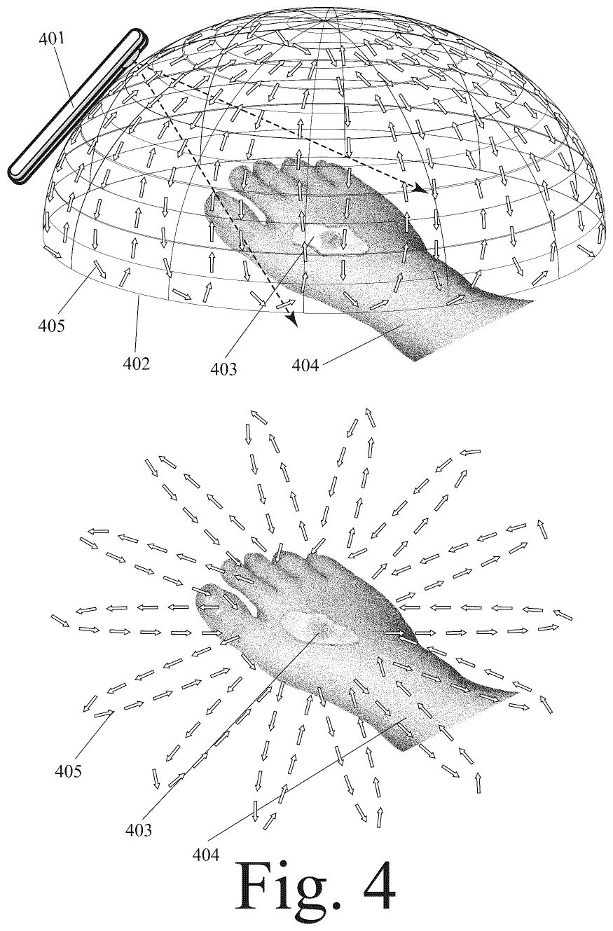

FIG. 4 shows an example of a method, device, and/or system for guiding a person concerning how to move a phone (or other imaging device) to record images of a pathology or injury (e.g. a wound, ulcer, or skin lesion) on a portion (e.g. a foot) of a body comprising: a phone (or other imaging device) 401; wherein the phone is configured to be moved along a virtual arcuate (e.g. undulating and/or starburst) path 405 on the surface of a virtual section 402 of a sphere (e.g. hemisphere) to record images of a pathology or injury 403 (e.g. wound, ulcer, or skin lesion) on the portion (e.g. foot) 404 of a body from different perspectives. In this example, the path along which the phone is moved is a undulating and/or starburst path along the surface of a section of a sphere (e.g. a hemisphere). In this example, this section is centered on a location on the pathology or injury. In this figure, the undulating and/or starburst path along which the phone travels is represented by a series of arrows.

The upper portion of FIG. 4 shows an oblique side view of this method, device, and/or system, including an oblique side view of the virtual starburst shape on the surface of a virtual hemisphere. The upper portion shows the phone (or other imaging device), the pathology or injury (e.g. wound, ulcer, or skin lesion), the portion of the body (e.g. foot), the section (e.g. hemisphere) of a sphere, and the virtual arcuate (e.g. undulating and/or starburst) path on the virtual hemisphere along which the phone is moved. The lower portion of FIG. 4 shows a top-down view of this method, device, and/or system. The lower portion does not include the phone or the hemisphere so that the undulating and/or starburst path can be seen more clearly.

In an example, a method corresponding to FIG. 4 for recording images of a pathology (or injury) on a body can comprise: (a) receiving a first set of one or more images of a pathology (or injury) on a portion of a body which have been recorded by a phone (or other imaging device); (b) using machine learning and/or artificial intelligence to analyze the first set of one or more images and to specify a virtual arcuate (e.g. undulating and/or starburst) path in space from which a second set of one or more images of the pathology should be recorded; and (c) guiding a person concerning how to move the phone along the virtual arcuate (e.g. undulating and/or starburst) path in space to record the second set of one or more images.

In an example, the second set of one or more images can be superior for medical evaluation purposes than the first set of one or more images. In an example, the second set of one or more images can correct problems with the first set of one or more images such as poor focus, limited field of view, limited range of angles, and/or poor illumination. In an example, the second set of images can be used to create a digital 3D model of the pathology (or injury) on a body. In an example, guidance concerning how to move the phone (or other imaging device) can be auditory (e.g. sounds, sound sequences, or spoken words). In an example, guidance concerning how to move the phone (or other imaging device) can be visual (e.g. directional indicators or words shown on a screen and/or in augmented reality). In an example, guidance concerning how to move the phone (or other imaging device) can be tactile (e.g. a vibration or sequence of vibrations). Relevant variations discussed elsewhere in this disclosure or in priority-linked disclosures can also be applied to this example.

I claim:

1. A method to guide a person concerning how to move or otherwise operate a phone or other imaging device to record images of a pathology or injury on a body comprising:
receiving a first set of one or more images of a pathology or injury on a body recorded by a phone or other imaging device;
analyzing the first set of one or more images; and
guiding a person concerning how to use the phone or other imaging device to record a second set of one or more images of the pathology or injury.

2. The method in claim 1 wherein the second set is more accurate and/or more complete for medical evaluation than the first set for one or more of the following reasons: the second set shows the pathology or injury in sharper focus; the second set shows the pathology with greater magnification; the second set shows the pathology or injury from a wider range of angles; the second set shows the pathology or injury from a greater range of distances; the second set includes a larger portion of the pathology or injury; the second set has better illumination; and the second set includes an environmental object near the pathology or injury for calibration of color and/or size.

3. The method in claim 1 wherein the person is guided concerning how to move or otherwise operate the phone or other imaging device by visual guidance comprising written words, arrows or other directional indictors, graphic symbols, animations, light emission, laser beams, and/or displayed images.

4. The method in claim 3 wherein the person is guided concerning how to move or otherwise operate the phone or other imaging device by an augmented reality display.

5. The method in claim 4 wherein the augmented reality display shows the pathology, the location of the phone or other imaging device, and how the phone or other imaging device should be moved.

6. The method in claim 1 wherein the person is guided concerning how to move or otherwise operate the phone or other imaging device by auditory guidance comprising spoken words, tones or sounds, and/or tone or sound sequences.

7. The method in claim 1 wherein the person is guided concerning how to move or otherwise operate the phone or other imaging device by tactile and/or haptic guidance comprising vibrations and/or vibration sequences.

8. The method in claim 1 wherein the person is given one or more auditory, visual, and/or tactile cues, signals, and/or communications with first characteristics if the phone or other imaging device should be moved closer to the pathology and the person is given one or more auditory, visual, and/or tactile cues, signals, and/or communications with second characteristics if the phone or other imaging device should be moved father from the pathology.

9. The method in claim 1 wherein machine learning and/or artificial intelligence is used for: analyzing the first set of one or more images; and guiding the person concerning how to move or otherwise operate the phone or other imaging device to record the second set of one or more images.

10. The method in claim 1 wherein: analysis of the first set of one or more images includes identifying a virtual path or shape in three-dimensional space which is across, over, or around the pathology along which the phone or other imaging device should be moved to record the second set of one or more images; and the person is guided concerning how to move the phone or other imaging device along the virtual path or shape.

11. The method in claim 10 wherein the virtual path or shape is a section of a circle, sphere, ellipse, or ellipsoid.

12. The method in claim 10 wherein the person is guided to move the phone in an oscillating, back-and-forth, sinusoidal, serpentine, spiral, helical, or starburst pattern along the virtual path or shape.

13. The method in claim 10 wherein the person is given one or more auditory, visual, and/or tactile cues, signals, and/or communications with first characteristics if the phone or other imaging device is on the virtual path or shape and the person is given one or more auditory, visual, and/or tactile cues, signals, and/or communications with second characteristics if the phone or other imaging device is not on the virtual path or shape.

14. A system to guide a person concerning how to use a phone or other imaging device to record images of a pathology or injury on a body comprising:
a phone or other imaging device which records images of a pathology or injury on a body; and
an augmented reality display.

15. The system in claim 14 wherein machine learning and/or artificial intelligence is used to analyze the images of the pathology or injury.

16. The system in claim 14 wherein machine learning and/or artificial intelligence is used to provide a person with guidance concerning how to move the phone or other imaging device to record images of the pathology or injury.

17. The system in claim 14 wherein guidance concerning how to move the phone or other imaging device is provided via the augmented reality display.

18. The system in claim 14 wherein the augmented reality display shows the pathology, the location of the phone or other imaging device, and how the phone or other imaging device should be moved.

19. The system in claim 14 wherein the augmented reality display is on the screen of a second phone or other mobile device.

20. The system in claim 14 wherein the augmented reality display is part of eyewear.
